(19) United States
(12) Patent Application Publication
(10) Pub. No.: US 2009/0035787 A1
(43) Pub. Date: Feb. 5, 2009

(54) ENZYMATIC SYNTHESIS OF SULFATED POLYSACCHARIDES WITHOUT IDURONIC ACID RESIDUES

(75) Inventors: Jian Liu, Chapel Hill, NC (US); Courtney Jones, Durham, NC (US); Jinghua Chen, Wuhan (CN); Yongmei Xu, Chapel Hill, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: The University of North Carolina at Chapel Hill

(21) Appl. No.: 12/178,434

(22) Filed: Jul. 23, 2008

Related U.S. Application Data
(60) Provisional application No. 60/961,716, filed on Jul. 23, 2007.

(51) Int. Cl. G01N 33/53; C08B 37/10; C12P 19/26; C12Q 1/02; C40B 50/06

(52) U.S. Cl. 435/7.1; 536/21; 536/122; 435/84; 506/26; 435/15; 435/29

(57) ABSTRACT

Iduronic acid (IdoUA)-free heparan sulfate (HS)-like compounds are provided. Also provided are methods of synthesizing IdoUA-free HS-like compounds. The methods can include providing at least one O-sulfotransferase (OST) enzyme and a reaction mixture comprising 3′-phosphoadenosine 5′-phosphosulfate (PAPS); and incubating a polysaccharide substrate with the at least one OST and the reaction mixture, whereby the HS-like compounds are synthesized. Also disclosed are methods of synthesizing a library of HS-like compounds and methods of determining the mechanism of activity of HS-like compounds.

### RELATED APPLICATIONS

This application claims the benefit of U.S. Provisional Patent Application Ser. No. 60/961,716, filed Jul. 23, 2007; the disclosure of which is incorporated herein by reference in its entirety.

### GOVERNMENT INTEREST

The presently disclosed subject matter was made with U.S. Government support under Grant No. AI50050 awarded by National Institutes of Health. Thus, the U.S. Government has certain rights in the presently disclosed subject matter.

### TECHNICAL FIELD

The presently disclosed subject matter relates generally to methods of sulfating polysaccharides. In some embodiments, the presently disclosed subject matter relates to methods of sulfating polysaccharides using O-sulfotransferases.

### BACKGROUND

Heparan sulfate (HS) is a ubiquitous component of the cell surface and extracellular matrix. It regulates a wide range of physiologic and pathophysiologic functions, including embryonic development and blood coagulation, and can facilitate viral infection (Esko and Selleck (2002) Annu. Rev. Biochem. 71, 435-471; Liu and Thorp (2002) Med. Res. Rev. 22, 1-25). HS exerts its biological effects by interacting with the specific proteins involved in a given process (Capila and Lindhardt (2002) Angew. Chem. Int Ed. 41, 390-412). HS is a highly charged polysaccharide comprising 1→4-linked glucosamine and glucuronic/iduronic acid units that contain both N- and O-sulfo groups. Unique saccharide sequences within HS determine the specificity of the binding of HS to its target proteins (Linhardt (2003) J. Med. Chem. 46, 2551-2564). Heparin, a specialized form of HS, is a commonly used anticoagulant drug. Thus, new methods for the synthesis of heparin and HS attract considerable interest for those developing anticoagulant and other HS-related drugs having improved pharmacological effects.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments of the presently disclosed subject matter, an iduronic acid (IdoUA)-free heparan sulfate (HS)-like compound comprising the following structure is provided:

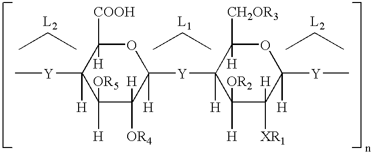

wherein X is NH or O, R₁ is H, SO₃H or CH₃, R₂ is SO₃H, R₃ is H or SO₃H, R₄ is H, SO₃H, CH₃ or CH₂CH₃, R₅ is H, SO₃H, CH₃ or CH₂CH₃, R₆ is COOH, COOCH₃, COOC₂H₅, CH₂OSO₃H, or CH₂SO₃H, L₁ is an alpha or beta linkage, L₂ is an alpha or beta linkage, Y is O or N, and n is ≧4. In some embodiments, the IdoUA-free HS-like compound is provided wherein X is NH, R₁ is SO₃H, R₂ is SO₃H, R₃ is SO₃H, R₄ is H or SO₃H, R₅ is H, L₁ is an alpha or a beta linkage, L₂ is an alpha or a beta linkage, and n is ≧4. In some embodiments, the IdoUA-free HS-like compound is provided wherein L₁ is an alpha linkage and L₂ is a beta linkage.

In some embodiments, the IdoUA-free HS-like compound has a binding affinity to antithrombin ranging from about 20 to about 60 nM. In some embodiments, the IdoUA-free HS-like compound has an anti-Xa activity ranging from about 10 to about 500 ng/ml. In some embodiments, the IdoUA-free HS-like compound has an anti-IIa activity ranging from about 5 to about 200 ng/ml. In some embodiments, the IdoUA-free HS-like compound has substantially reduced cell proliferation activity as compared to a HS-like compound with iduronic acid residues.

In some embodiments, the IdoUA-free HS-like compound is provided wherein R₄ is selected from the group consisting of CH₃, CH₂CH₃ and SO₃H and R₅ is selected from the group consisting of CH₃, CH₂CH₃ and SO₃H. In some embodiments, the IdoUA-free HS-like compound is provided wherein X is O and R₁ is SO₃H. In some embodiments, the IdoUA-free HS-like compound is provided wherein L₁ is an alpha linkage and L₂ is an alpha linkage. In some embodiments, the IdoUA-free HS-like compound is provided wherein L₁ is a beta linkage and L₂ is a beta linkage.

In some embodiments of the presently disclosed subject matter, a method of synthesizing an IdoUA-free HS-like compound is provided, comprising incubating a polysaccharide substrate with a sulfo donor compound and an enzyme mixture, the enzyme mixture comprising O-sulfotransferase (OST) enzymes 6-OST-1,6-OST-3 and 3-OST-1 and substantially no epimerase enzyme, wherein synthesis of the IdoUA-free HS-like compound from the polysaccharide substrate is accomplished. In some embodiments, the method comprises a polysaccharide substrate selected from the group consisting of N-sulfo heparosan and chemically desulfated N-sulfated heparin. In some embodiments, the OST enzymes are recombinant OST enzymes. In some embodiments, the recombinant OST enzymes are produced in a bacterial expression system. In some embodiments, the sulfo donor compound comprises a compound capable of donating a sulfonate or sulfuryl group. In some embodiments, the polysaccharide substrate is partially sulfated prior to incubation.

In some embodiments of the presently disclosed subject matter, the method of synthesizing an IdoUA-free HS-like compound further comprises (a) providing a reaction mixture comprising adenosine 3′,5′-diphosphate (PAP), a PAPS regenerating enzyme and a sulfo donor compound; and (b) incubating the reaction mixture for a time period sufficient to catalyze the production of 3′-phosphoadenosine 5′-phosphosulfate (PAPS) from the PAP by the PAPS regenerating enzyme utilizing the sulfo donor compound as a sulfo substrate, whereby the sulfo donor compound is provided. In some embodiments, the PAPS regenerating enzyme is an arylsulfotransferase. In some embodiments, the arylsulfotransferase is AST-IV. In some embodiments, the PAPS regenerating enzyme is an estrogen sulfotransferase. In some embodiments, the sulfo donor compound is an aryl sulfate compound. In some embodiments, the aryl sulfate compound is p-nitrophenol sulfate (PNPS). In some embodiments, the time period is from about 1 minute to about 30 minutes. In some embodiments, the OST enzymes further comprise 2-OST. In some embodiments, the 2-OST enzyme is a recombinant OST enzyme. In some embodiments, the recombinant OST enzyme is produced in a bacterial expression system.

In some embodiments of the presently disclosed subject matter, a method of synthesizing a library of HS-like compounds is provided, comprising incubating polysaccharide substrates with a sulfo donor compound and one or more combinations of biosynthetic enzymes, the biosynthetic enzymes comprising epimerases, N-sulfotransferases and O-sulfotransferases. In some embodiments, the polysaccharide substrate is N-sulfo heparosan. In some embodiments, the O-sulfotransferases are selected from the group consisting of 2-OSTs, 3-OSTs and 6-OSTs. In some embodiments, the O-sulfotransferase enzymes are recombinant OST enzymes. In some embodiments, the recombinant OST enzymes are produced in a bacterial expression system. In some embodiments, the polysaccharide substrate is partially sulfated prior to incubation.

In some embodiments of the presently disclosed subject matter, the method of synthesizing a library of HS-like compounds further comprises (a) providing a reaction mixture comprising adenosine 3′,5′-diphosphate (PAP), a PAPS regenerating enzyme and a sulfo donor compound; and (b) incubating the reaction mixture for a time period sufficient to catalyze the production of 3′-phosphoadenosine 5′-phosphosulfate (PAPS), whereby the sulfo donor compound is provided. In some embodiments, the PAPS regenerating enzyme is an arylsulfotransferase. In some embodiments, the arylsulfotransferase is AST-IV. In some embodiments, the PAPS regenerating enzyme is an estrogen sulfotransferase. In some embodiments, the sulfo donor compound is an aryl sulfate compound. In some embodiments, the aryl sulfate compound is p-nitrophenol sulfate (PNPS). In some embodiments, the time period is from about 1 minute to about 30 minutes. In some embodiments, the polysaccharide substrate is a chemically desulfated N-sulfated heparin.

In some embodiments of the presently disclosed subject matter, a method of determining a mechanism of activity of HS-like compounds is provided, comprising (a) synthesizing one or more HS-like compounds by incubating one or more polysaccharide substrates with a sulfo donor compound and one or more combinations of biosynthetic enzymes, the biosynthetic enzymes comprising epimerases, N-sulfotransferases and O-sulfotransferases; (b) subjecting one or more of the HS-like compounds to a test for an activity; and (c) determining a mechanism of activity of the HS-like compounds based on one or more results of the one or more HS-like compounds. In some embodiments, the polysaccharide substrate is selected from the group consisting of N-sulfo heparosan and chemically desulfated N-sulfated heparin. In some embodiments, the O-sulfotransferases are selected from the group consisting of 2-OSTs, 3-OSTs and 6-OSTs. In some embodiments, the O-sulfotransferase enzymes are recombinant OST enzymes. In some embodiments, the recombinant OST enzymes are produced in a bacterial expression system. In some embodiments, the polysaccharide substrate is partially sulfated prior to incubation. In some embodiments, the test is for an activity selected from the group consisting of anti-thrombin binding activity, anti-Xa activity, anti-IIa activity, cell proliferation stimulation activity, cell growth stimulation activity, activated partial thromboplastin time, prothrombin time and combinations thereof.

In some embodiments of the presently disclosed subject matter, the method of determining a mechanism of activity of HS-like compounds further comprises (a) providing a reaction mixture comprising adenosine 3′,5′-diphosphate (PAP), a PAPS regenerating enzyme and a sulfo donor compound; and (b) incubating the reaction mixture for a time period sufficient to catalyze the production of 3′-phosphoadenosine 5′-phosphosulfate (PAPS), whereby the sulfo donor compound is provided. In some embodiments, the PAPS regenerating enzyme is an arylsulfotransferase. In some embodiments, the arylsulfotransferase is AST-IV. In some embodiments, the PAPS regenerating enzyme is an estrogen sulfotransferase. In some embodiments, the sulfo donor compound is an aryl sulfate compound. In some embodiments, the aryl sulfate compound is p-nitrophenol sulfate (PNPS). In some embodiments, the time period is from about 1 minute to about 30 minutes.

Accordingly, it is an object of the presently disclosed subject matter to provide iduronic acid-free heparan sulfate-like compounds. Further, it is an object of the presently disclosed subject matter to provide methods of synthesizing iduronic acid-free heparan sulfate-like compounds. These objects are achieved in whole or in part by the presently disclosed subject matter.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIGS. 1A and 1B are schematic drawings illustrating the biosynthesis of HS-like compounds. FIG. 1A is a schematic drawing of the biosynthetic pathway of HS. FIG. 1B is a schematic drawing of the synthesis of a library of HS-like compounds using an enzyme-based combinatorial approach as disclosed herein. Each of compounds 1-8 are non-limiting examples of HS-like compounds prepared using the disclosed enzyme-based combinatorial approach. The enzyme schemes used for the synthesis of each of compounds 1-8 are shown in Table 2.

FIGS. 2A and 2B are reversed-phased ion pairing (RPIP) HPLC chromatograms of the disaccharide analysis of compounds 8 (FIG. 2A) and 2 (FIG. 2B). The ³⁵S-labeled polysaccharides were degraded by nitrous acid at pH 1.5 followed by sodium borohydride reduction. The resultant ³⁵S-labeled disaccharides were desalted on a BIO-GEL® P-2 column, and then resolved by RPIP-HPLC chromatography. The elution positions were identified by co-eluting with ³⁵S-labeled or ³H-labeled disaccharide standards, where peak label 1 represents GlcUA-AnMan3S, 2 represents IdoUA2S-AnMan, 3 represents GlcUA-AnMan6S, 4 represents IdoUA-AnMan6S, 5 represents IdoUA-AnMan3S6S, 6 represents GlcUAAnMan3S6S, and 7 represents IdoUA2S-AnMan6S.

FIG. 3 is a graph showing the effect of the synthetic HS-like compounds on FGF-2-dependent BaF3 FGFR1c cell proliferation. BaF3 FGFR1c cells were seeded in 96-well plates with 2 nM FGF2 for control and 2 nM FGF2 plus various concentrations of the following compounds: heparin, 1, 2, 7 and 8. Cells were cultured for 40 hrs, followed by incubation in the media containing [³H]thymidine for 4 hrs. The cellular proliferation was determined by [³H]thymidine incorporation into the DNA. Data are mean± range of duplicates. In the graph, heparin is represented by a solid line and solid circle, compound 8 by a dotted line and open circle, compound 2 by a short dashed line and solid circle, compound 1 by a dashed and dotted line and open circle, and compound 7 by a broken line and solid circle.

FIG. 4 shows the elution profiles of N-[³⁵S]sulfo heparosan oligosaccharides on BIO-GEL® P-10 column. Low specific [³⁵S]radioactively labeled N-[³⁵S]sulfo heparosan (1.5×10⁶ cpm/mg) was prepared by incubating deacetylated heparosan with purified NST and [³⁵S]PAPS. The polysaccharide (1 mg) was digested with 20 ng of purified heparin lyase Ill. The products were fractionated on a Bio-Gel®™ P-10 column, which was eluted with a buffer containing 25 mM Tris, 1000 mM NaCl, pH 7.4. The fractions were monitored by both [³⁵S]radioactivity and UV 232 nm. In the graph, [³⁵S]radioactivity is indicated on the left vertical axis and represented by the black line and black dots, while UV optical density (O.D.) at 232 nm is indicated on the right vertical axis and represented by the gray line and gray dots. The fractions containing the oligosaccharides with the desired size were pooled for further analysis.

FIG. 5 is a bar graph showing the enzymatic synthesis of AT-binding oligosaccharides. The oligosaccharide substrates (1 μg) or the full length N-sulfo heparosan (1 μg) were incubated with a mixture of enzymes, including 6-OST-1 (20 μg), 6-OST-3 (20 μg) and 3-OST-1 (10 μg) and [³⁵S]PAPS (30 μM, 285,000 cpm/nmole) at 37° C. for 2 hrs. Alternatively, the oligosaccharides (1 μg) or full length N-sulfo heparosan (1 μg) were preincubated with epimerase (8 μg) at 37° C. for 30 min followed by incubating with mixture of 6-OST-1 (20 μg), 6-OST-3 (20 μg), 2-OST (40 μg) and 3-OST-1 (10 μg) in the presence of [³⁵S]PAPS. The resultant ³⁵S-labeled oligosaccharides were incubated with AT, and the complex of AT and ³⁵S-labeled oligosaccharides were captured by ConA-agarose column. The amount of ³⁵S-labeled products using full length N-sulfo heparosan was defined to be 100%. In the graph, the ³⁵S-labeled oligosaccharides synthesized in the absence of epimerase and 2-OST are represented by the solid black bars while the ³⁵S-labeled oligosaccharides synthesized with epimerase and 2-OST are represented by the solid gray bars.

FIG. 6 is a schematic illustration of the synthetic scheme of compound 2 (Recomparin). The components involved in PAPS regeneration system are boxed.

FIGS. 7A and 7B are graphs illustrating the inhibitory effects of heparin (FIG. 1A) and recomparin (compound 2; FIG. 6), respectively, on Xa activity. Heparin and recomparin were incubated with AT (20 μg/ml), factor Xa (10 U/ml) and bovine serum albumin (100 μg/ml) in 20 mM sodium phosphate and 150 mM NaCl, pH 7.2, and 1 mM S-2765 chromogenic Xa substrate. The activity of Xa was determined by the rate of the increase of the absorbance at 405 nm. The activity without polysaccharide was defined as 100%. Each data point represents the average of two determinations. Error bars indicate the range.

### DETAILED DESCRIPTION

Throughout the specification and claims, a given chemical formula or name shall encompass all optical isomers and stereoisomers, as well as racemic mixtures where such isomers and mixtures exist.

### I. General Considerations

Heparan sulfates (HSs) are highly sulfated polysaccharides present on the surface of mammalian cells and in the extracellular matrix in large quantities. HS is a highly charged polysaccharide comprising 1→4-linked glucosamine and glucuronic/iduronic acid units that contain both N- and O-sulfo groups. Heparin, a specialized form of HS, is a commonly used anticoagulant drug. Thus, “heparan sulfate”, as used herein, includes heparin.

As used herein, the terms “HS-like compound” and “HS-like molecule” are intended to refer to synthetically sulfated polysaccharides possessing one or more structural and/or functional properties of HSs. In some embodiments, HS-like compounds can contain glucuronic acid or iduronic acid and glucosamine with or without sulfo groups. As such, HS-like compounds and HS-like molecules include, but are not limited to, synthetic HSs, sulfated polysaccharides and heparins. Although exemplary embodiments of particular HS-like compounds or molecules have been disclosed herein, the presently disclosed subject matter is not intended to be limited to the disclosed examples, but rather HS-like compounds and HS-like molecules include all comparable synthetically sulfated polysaccharides as would be apparent to one of ordinary skill. Indeed, one of ordinary skill in the art, upon a review of the instant disclosure, is capable of producing numerous HS-like compounds based upon the disclosed methods and compounds.

HSs play critical roles in a variety of important biological processes, including assisting viral infection, regulating blood coagulation and embryonic development, suppressing tumor growth, and controlling the eating behavior of test subjects by interacting with specific regulatory proteins (Liu, J., and Thorp, S. C. (2002) Med. Res. Rev. 22:1-25; Rosenberg, R. D., et al., (1997) J. Clin. Invest. 99:2062-2070; Bernfield, M., et al., (1999) Annu. Rev. Biochem. 68:729-777; Alexander, C. M., et al., (2000) Nat. Genet. 25:329-332; Reizes, O., et al., (2001) Cell 106:105-116). The unique sulfated saccharide sequences determine to which specific proteins HSs bind, thereby regulating biological processes.

The biosynthesis of HS occurs in the Golgi apparatus. It is initially synthesized as a copolymer of glucuronic acid and N-acetylated glucosamine by D-glucuronyl and N-acetyl-D-glucosaminyltransferase, followed by various modifications (Lindahl, U., et al., (1998) J. Biol. Chem. 273:24979-24982). These modifications include N-deacetylation and N-sulfation of glucosamine, C₅ epimerization of glucuronic acid to form iduronic acid residues, 2-O-sulfation of iduronic and glucuronic acid, as well as 6-O-sulfation and 3-O-sulfation of glucosamine. Several enzymes that are responsible for the biosynthesis of HS have been cloned and characterized (Esko, J. D., and Lindahl, U. (2001) J. Clin. Invest 108:169-173).

The expression levels of various HS biosynthetic enzyme isoforms contribute to the synthesis of specific saccharide sequences in specific tissues. HS N-deacetylase/N-sulfotransferase, 3-O-sulfotransferase, and 6-O-sulfotransferase are present in multiple isoforms. Each isoform is believed to recognize a saccharide sequence around the modification site in order to generate a specific sulfated saccharide sequence (Liu, J., et al., (1999) J. Biol. Chem. 274:5185-5192; Aikawa, J.-I., et al., (2001) J. Biol. Chem. 276:5876-5882; Habuchi, H., et al., (2000) J. Biol. Chem. 275:2859-2868). For instance, HS D-glucosaminyl 3-O-sulfotransferase (3-OST) isoforms generate 3-O-sulfated glucosamine residues that are linked to different sulfated iduronic acid residues. 3-OST isoform 1 (3-OST-1) transfers sulfate to the 3-OH position of an N-sulfated glucosamine residue that is linked to a glucuronic acid residue at the nonreducing end (GlcUA-GlcNS±6S). However, 3-OST isoform 3 (3-OST-3) transfers sulfate to the 3-OH position of an N-unsubstituted glucosamine residue that is linked to a 2-O-sulfated iduronic acid at the nonreducing end (IdoUA2S-GlcNH₂±6S) (Liu, J., et al., (1999) J. Biol. Chem. 274:38155-38162). The difference in the substrate specificity of 3-OSTs results in distinct biological functions. For example, the HS modified by 3-OST-1 binds to antithrombin (AT) and possesses anticoagulant activity (Liu, J., et al., (1996) J. Biol. Chem. 271:27072-27082). However, the HS modified by 3-OST-3 (3-OST-3A and 3-OST-3B) binds to glycoprotein D (gD) of herpes simplex virus, type 1, (HSV-1) thus mediating viral entry (Shukla, D., et al., (1999) Cell 99:13-22).

Cell surface HS also assists HSV-1 infection (WuDunn, D., and Spear, P. G. (1989) J. Virol. 63:52-58). One report (Shukla, D., et al., (1999) Cell 99:13-22) suggests that a specific 3-O-sulfated HS is involved in assisting HSV-1 entry. The 3-O-sulfated HS is generated by 3-OST-3 but not by 3-OST-1. In addition, the 3-O-sulfated HS provides binding sites for HSV-1 envelope glycoprotein D, which is a key viral protein involved in the entry of HSV-1 (Shukla, D., et al., (1999) Cell 99:13-22). Because 3-OST-3-modified HS is rarely found in HS from natural sources, the study suggests that HSV-1 recognizes a unique saccharide structure. Indeed, the result from the structural characterization of a gD-binding octasaccharide revealed that the octasaccharide possesses a specific saccharide sequence (Liu, J., et al., (2002) J. Biol. Chem. 277:33456-33467). In addition, the binding affinity of the 3-O-sulfated HS for gD is about 2 μM (Shukla, D., et al., Cell 99:13-22). This affinity is similar to that reported for the binding of gD to the protein receptors, suggesting that HSV-1 utilizes both protein and HS cell surface receptors to infect target cells (Willis, S. H., et al., (1998) J. Virol. 72:5938-5947; Krummenacher, C., et al., (1999) J. Virol. 73:8127-8137). It is believed that the interaction between gD and the 3-O-sulfated the protein entry receptors somehow triggers the fusion between the virus and the cell in the presence of other viral envelope proteins, including gB, gH, and gL (Shukla, D., and Spear, P. G. (2001) J. Clin. Invest. 108:503-510). A study of the co-crystal structure of gD and herpes entry receptor HveA suggests that the binding of HveA to gD induces conformational changes in gD (Carfi, A., et al., (2001) Mol. Cell. 8:169-179).

HS-regulated anticoagulation mechanisms have been studied extensively. It is now known that HS, including heparin, interact with AT, a serine protease inhibitor, to inhibit the activities of thrombin and factor Xa in the blood coagulation cascade (Rosenberg, R. D., et al., (1997) J. Clin. Invest. 99:2062-2070). Anticoagulant-active HS(HS^{act}) and heparin contain one or multiple AT-binding sites per polysaccharide chain. This binding site contains a specific pentasaccharide sequence with a structure of -GlcNS(or Ac)6S-GlcUA-GlcNS3S(±6S)-IdoUA2S-GlcNS6S-. The 3-O-sulfation of glucosamine for generating GlcNS3S(+6S) residue, which is carried out by 3-OST-1, plays a role in the synthesis of HS^{act} (Liu, J., et al., (1996) J. Biol. Chem. 271:27072-27082; Shworak, N. W., et al., (1997) J. Biol. Chem. 272:28008-28019).

In accordance with some embodiments of the presently disclosed subject matter, HS-like compounds can have a strong binding affinity for AT. By way of non-limiting example, the binding constant (K_{d}) of an HS-like molecule can range from about 5 to about 100 nM. In some embodiments, the binding constant (K_{d}) of an HS-like molecule can range from about 20 to about 60 nM. Any suitable approach to determine binding affinity can be employed as would be appreciated by one of ordinary skill in the art upon review of the instant disclosure.

In some embodiments the anticoagulant activity can be measured by determining anti-Xa and anti-IIa activities. In some embodiments, the anti-Xa and anti-IIa activities can be determined in the presence of antithrombin. HS-like compounds with a strong binding affinity for AT and/or a high anticoagulant activity can have high anti-Xa and anti-IIa activities. In some embodiments, HS-like compounds with high anticoagulant activity can have IC₅₀ values for anti-Xa activity ranging from about 10 to about 500 ng/ml. In some embodiments, HS-like compounds with high anticoagulant activity can have IC₅₀ values for anti-Xa activity ranging from about 20 to about 100 ng/ml. In some embodiments, HS-like compounds with high anticoagulant activity can have IC₅₀ values for anti-IIa activity ranging from about 5 to about 200 ng/ml. In some embodiments, HS-like compounds with high anticoagulant activity can have IC₅₀ values for anti-IIa activity ranging from about 5 to about 50 ng/ml. Any suitable approach to determine anticoagulant activity can be employed as would be appreciated by one of ordinary skill in the art upon review of the instant disclosure.

### II. Methods of Sulfating Polysaccharides

The presently disclosed subject matter provides in some embodiments enzymatic methods for the sulfation of multimilligram amounts of HS-like compounds having particular functions, using sulfotransferases coupled with a system for reducing inhibitory effects from sulfo donor byproducts. In some embodiments, the system for reducing inhibitory byproducts comprises a 3′-phosphoadenosine 5′-phosphosulfate (PAPS) regeneration system. In some embodiments, the PAPS regeneration system can utilize arylsulotransferase IV, while in some embodiments estrogen sulfotransferase is employed. In some embodiments, the system comprises a phosphatase enzyme. By utilizing the presently disclosed sulfation system and selecting appropriate enzymatic modification steps, an inactive precursor polysaccharide can be converted to a HS-like compound having desired biological properties.

In some embodiments, the presently disclosed subject matter employs recombinant sulfotransferases. Because the recombinant sulfotransferases can be recombinantly expressed in bacteria, and the disclosed methods can use low cost sulfo donors, the presently disclosed subject matter can be readily utilized to synthesize large quantities of biologically active heparan sulfates while reducing the production of reaction inhibitory byproducts.

Two representative advantages provided by the presently disclosed subject matter facilitate the large scale synthesis of HS. First, large amounts of all the required HS sulfotransferases can be successfully recombinantly expressed in Escherichia coli. Second, the enzymatic sulfation reactions can be coupled with a system for reducing inhibitory effects from sulfo donor byproducts (e.g., PAP) and reducing costs related to continuously providing a supply of the sulfo donor PAPS. PAPS, a universal sulfate donor and source of sulfate for all sulfotransferases, is a highly expensive and unstable molecule that has been an obstacle to the large-scale production of enzymatically sulfated products. The half-life of PAPS in aqueous solution at pH 8.0 is approximately 20 hours. Product inhibition by adenosine 3′,5′-diphosphate (PAP) has also been a limiting factor to large-scale applications. For example, PAP inhibition of hydroxysteroid sulfotransferase was determined to be Kᵢ=14 μM (Marcus et al. (1980) Anal. Biochem. 107, 296). PAP has also been shown to inhibit the sulfotransferase NodST with a Kᵢ=0.1 μM (Lin et al., (1995) J. Am. Chem. Soc. 117, 8031). In some embodiments of the presently disclosed subject matter, a PAPS regeneration system, such as the system developed by Burkart and colleagues (Burkart et al. (2000) J. Org. Chem. 65, 5565-5574, incorporated herein by reference), has been modified and adapted to be coupled to the enzymatic synthesis reactions. The PAPS regeneration system converts PAP into PAPS, thereby reducing accumulation of inhibitory PAP in the reaction mixture and reducing production costs related to providing PAPS to drive the sulfation reaction. In other embodiments, phosphatase enzymes can be utilized to modify PAP so that it no longer has binding affinity for sulfotransferases.

The presently disclosed sulfation system can be adapted to produce a multitude of HS-like compounds having varied biological activities by selecting appropriate sulfotransferases to include and by controlling (for example, sequentially controlling) the addition of those sulfotransferases to the reaction system to facilitate appropriate timing of sulfations of the polysaccharide template. For example, utilizing the presently disclosed methods, HS-like compounds can be synthesized with purposeful and specific biological activities, such as but not limited to: anticoagulant HS, fibroblast growth factor-2-binding activity, herpes simplex virus glycoprotein D (gD)-binding HS, and fibroblast growth factor 2 (FGF2) receptor-binding HS. For example, the enzymatic synthesis of a gD-binding octasaccharide can be used to inhibit HSV-1 infection (Copeland et al (2008) Biochemistry 47: 5774-5783).

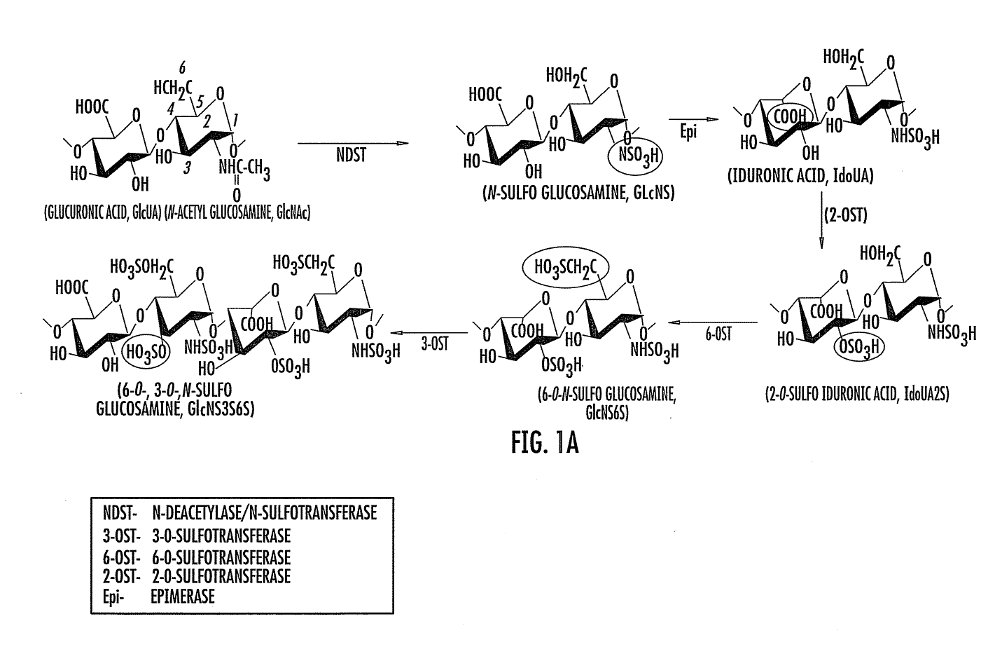

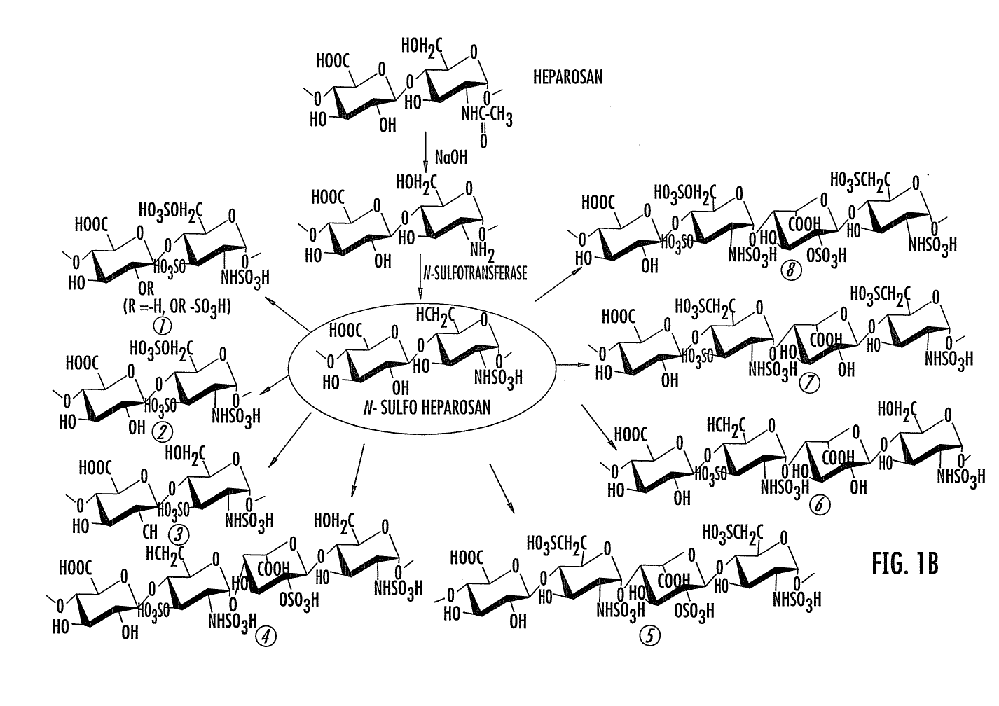

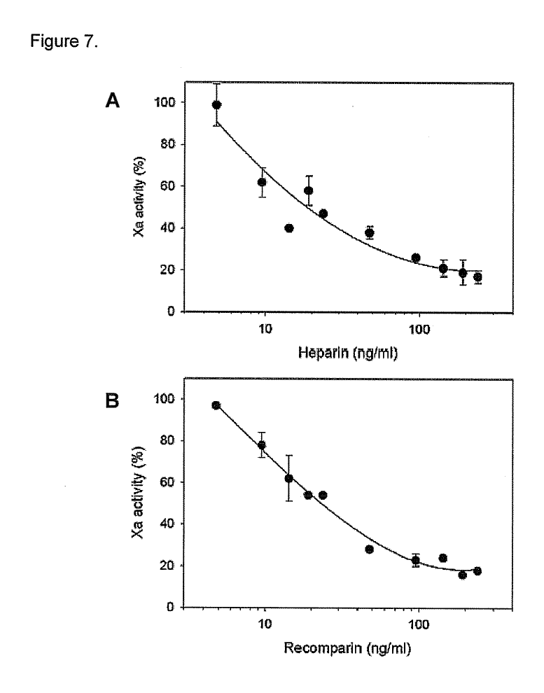

By way of example and not limitation, few as two or three enzymatic steps are required for the synthesis of each of these biologically-active HS molecules (See, e.g., FIGS. 1A, 1B and 7). Thus, the presently disclosed subject matter provides for the large scale synthesis of a wide range of HS-like compounds with specific biological activities. In addition, provided in some embodiments is a model system to better understand the biosynthesis and the functional components of HS-like compounds.

In some embodiments of the presently disclosed subject matter, a method of sulfating a polysaccharide is provided. In some embodiments, the method comprises incubating a polysaccharide substrate to be sulfated with a reaction mixture that comprises at least one sulfotransferase enzyme, such as for example an O-sulfotransferase (OST) enzyme, and a sulfo donor, such as for example PAPS. When PAPS is employed, production of the sulfated polysaccharide from the polysaccharide substrate is catalyzed by the OST enzyme with a conversion of the PAPS to adenosine 3′,5′-diphosphate (PAP). A reaction condition is further provided that modifies generated PAP to reduce an inhibitory effect of PAP on the polysaccharide sulfation. For example, a PAPS regeneration system can be coupled with the sulfation reaction to convert PAP into PAPS, or phosphatases can be added to the reaction mixture to modify PAP such that it does not compete with PAPS for binding with OSTs.

In some embodiments, the polysaccharide substrate is a previously N,O-desulfated and subsequently re-N-sulfated polysaccharide, such as for example a chemically desulfated N-sulfated (CDSNS) heparin. In other embodiments, the polysaccharide substrate is partially sulfated prior to reaction mixture incubation. For example, a CDSNS can be reacted with a particular OST to produce a sulfated polysaccharide intermediate product that can then be reacted with one or more different OSTs to further sulfate the polysaccharide at different locations. This sequential process of reacting the polysaccharide substrate with one or more different OSTs can be continued until a final polysaccharide is produced exhibiting desired biological activities (based, at least in part, on the sulfation pattern of the polysaccharide).

In some embodiments the sulfated polysaccharide product can be a glycosaminoglycan (GAG). GAGs are the most abundant heteropolysaccharides in the body. These molecules are long unbranched polysaccharides containing a repeating disaccharide unit. The disaccharide units can contain either of two modified sugars: N-acetylgalactosamine (GalNAc) or N-acetylglucosamine (GlcNAc) and a uronic acid such as glucuronate or iduronate. GAGs are highly negatively charged molecules, with extended conformation that imparts high viscosity to the solution. Along with the high viscosity of GAGs comes low compressibility, which makes these molecules ideal for a lubricating fluid in the joints. At the same time, their rigidity provides structural integrity to cells and provides passageways between cells, allowing for cell migration. The specific GAGs of physiological significance are hyaluronic acid, dermatan sulfate, chondroitin sulfate, heparin, heparan sulfate (including heparin), and keratan sulfate. Thus, in some embodiments, the sulfated polysaccharide product is a HS. In some embodiments, the sulfated polysaccharide product is an anticoagulant-active HS, an antithrombin-binding HS, an FGF-binding HS, and an HSV gD-binding HS.

FIG. 1B schematically illustrates the enzyme-based combinatorial approach to synthesizing HS-like compounds by selectively reacting a polysaccharide substrate with one or more different OSTs and biosynthetic enzymes (Table 2). For example, compound 1 can be produced by incubating a polysaccharide substrate (e.g., N-sulfo heparosan) with 2-OST, 6-OST-1,6-OST-3 and 3-OST-1, wherein the reaction is substantially free of epimerase. Compound 1 has no iduronic acid residue. Alternatively, incubating a polysaccharide substrate with 6-OST-1,6-OST-3 and 3-OST-1 produces compound 2, which has no iduronic acid residue or 2-O-sulfo group. Further, compound 3 can be synthesized by reacting a polysaccharide substrate with 3-OST-1 and 2-OST, while incubating a polysaccharide substrate with epimerase in addition to 3-OST-1 and 2-OST yields compound 4. Compound 3 has no iduronic acid residue and neither of compounds 3 or 4 have 6-O-sulfation. This enzyme-based combinatorial approach was used to produce compounds 5-8 in a similar manner, as illustrated in FIG. 1B and summarized in Table 2.

As illustrated in FIG. 1B and summarized in Table 2, the reactions described above for the production of compounds 1, 2 and 3 are substantially free of epimerase. The absence of epimerase in these reactions results in the production of IdoUA-free compounds.

Formula 1 is a structural rendering of the basic structural unit of HS-like compounds or molecules of the instant disclosure, which can be synthesized by the presently disclosed methods, including but not limited to the disclosed enzyme-based combinatorial approach. Formula 1 illustrates the basic structural unit of the HS-like compounds of the instant disclosure as well as non-limiting examples of the variable structural features such as variable R groups. In some embodiments the basic structural unit of the HS-like compound illustrated in Formula 1 generally comprises a glucuronic acid residue or an iduronic acid residue linked to a glucosamine residue. In some embodiments the glucuronic acid or iduronic acid residue can be linked to the glucosamine by an alpha linkage, represented by L₁ in Formula 1. In some embodiments the glucuronic acid or iduronic acid residue can be linked to the glucosamine by a beta linkage, represented by L₁ in Formula 1.

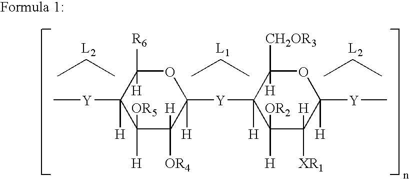

X═NH or O

R₁═H, SO₃H, or CH₃

R₂═SO₃H

R₃═H or SO₃H

R₄═H, SO₃H, CH₃ or CH₂CH₃

R₅═H, SO₃H, CH₃ or CH₂CH₃

R₆═COOH, COOCH₃, COOC₂H₅, CH₂OSO₃H or CH₂SO₃H

Y═O or N

L₁=alpha linkage or beta linkage

L₂=alpha linkage or beta linkage

The basic structural unit of HS-like compounds illustrated in Formula 1 can be repeated a plurality of times to form a linear copolymer of glucuronic acid or iduronic acid and glucosamine residues, resulting in the series of basic structural units which form the general structure of the HS-like compound. In Formula 1 the brackets with subscript n ([ ]ₙ) represent the repetitive nature of this linear chain of basic structural units of glucuronic acid or iduronic acid and glucosamine residues. In some embodiments, the basic structural unit of the HS-like compound is linked to one or more further basic structural units by an alpha linkage, represented by L₂, to form the linear chain. In some embodiments, the basic structural unit of the HS-like compound is linked to one or more further basic structural units by a beta linkage, represented by L₂, to form the linear chain. Accordingly, an HS-like compound of the presently disclosed subject matter, as illustrated in Formula 1, can comprise L₁ alpha linkages and L₂ beta linkages. Alternatively, in some embodiments an HS-like compound as illustrated in Formula 1 can comprise L₁ beta linkages and L₂ alpha linkages. Further yet, in some embodiments an HS-like compound as illustrated in Formula 1 can comprise both L₁ and L₂ alpha linkages, or alternatively, both L₁ and L₂ beta linkages. By way of non-limiting example, the alpha and beta linkages described herein can comprise alpha 1→4 (α1→4) or beta (β1→4) linkages. The L₁ and L₂ linkages can comprise O or N molecules. In some embodiments both L₁ and L₂ can comprise O molecules. In some embodiments both L₁ and L₂ can comprise N molecules. In some embodiments L₁ can comprise an O molecule and L₂ a N molecule. Still yet, in other embodiments, L₁ can comprise a N molecule and L₂ an O molecule. Further, in some embodiments, L₁ and L₂ can comprise any combination of alpha and beta linkages and O and N molecules.

There appears to be no known limit to the length of the linear copolymer of basic structural units which form the general structure of the HS-like compound. Referring again to Formula 1, in some embodiments, n is ≧1. In some embodiments, n is ≧2. In some embodiments, n is ≧3. In some embodiments, n is ≧4. In some embodiments, n is ≧5. In some embodiments, n is ≧10. In some embodiments, n is ≧20. In some embodiments, n is ≧100.

In some embodiments, the HS-like molecule illustrated in Formula 1 has as group X an NH or O. In some embodiments, R₁ can be H or SO₃H. In some embodiments, R₂ can be SO₃H. In some embodiments, R₃ can be H or SO₃H. In some embodiments, R₄ can be H, SO₃H, CH₃ or CH₂CH₃. In some embodiments, R₅ can be H, SO₃H, CH₃ or CH₂CH₃. In some embodiments, R₆ can be COOH, COOCH₃, COOC₂H₅, CH₂OSO₃H or CH₂SO₃H.

In some embodiments, an HS-like compound of the presently disclosed subject matter can comprise an HS-like compound as illustrated in Formula 1, wherein X is NH or O, R₁ is H or SO₃H, R₂ is SO₃H, R₃ is H or SO₃H, R₄ is H, SO₃H, CH₃ or CH₂CH₃, R₅ is H, SO₃H, CH₃ or CH₂CH₃, R₆ is COOH, COOCH₃, COOC₂H₅, CH₂OSO₃H, or CH₂SO₃H, L₁ is an alpha or beta linkage, L₂ is an alpha or beta linkage, and n is ≧4. In some embodiments, an HS-like compound of the presently disclosed subject matter can comprise an HS-like compound as illustrated in Formula 1, wherein X is NH, R₁ is SO₃H, R₂ is SO₃H, R₃ is SO₃H, R₄ is H or SO₃H, R₅ is H, L₁ is a beta linkage, L₂ is an alpha linkage, and n is ≧4. In some embodiments, an HS-like compound of the presently disclosed subject matter can comprise an HS-like compound as illustrated in Formula 1, wherein X is NH, R₁ is SO₃H, R₂ is SO₃H, R₃ is SO₃H, R₄ is H or SO₃H, R₅ is H, L₁ is an alpha linkage, L₂ is a beta linkage, and n is ≧4. In some embodiments, an HS-like compound of the presently disclosed subject matter can comprise an HS-like compound as illustrated in Formula 1, wherein X is NH, R₁ is SO₃H, R₂ is SO₃H, R₃ is SO₃H, R₄ is H or SO₃H, R₅ is H, L₁ is an alpha linkage, L₂ is an alpha linkage, and n is ≧4. In some embodiments, an HS-like compound of the presently disclosed subject matter can comprise an HS-like compound as illustrated in Formula 1, wherein X is NH, R₁ is SO₃H, R₂ is SO₃H, R₃ is SO₃H, R₄ is H or SO₃H, R₅ is H, L₁ is a beta linkage, L₂ is a beta linkage, and n is ≧4. In some embodiments, the HS-like compounds described herein are IdoUA-free. In some embodiments the HS-like compounds described herein are isolated and purified. In some embodiments, the HS-like compounds described herein are synthetically synthesized.

In some embodiments, the disclosed HS-like compounds can be synthesized chemically. Still yet, in some embodiments the disclosed HS-like compounds can be synthesized using a combination of enzymatic and chemical methods.

II.A. Sulfotransferases

As previously noted, the presently disclosed subject matter utilizes sulfotransferases, particularly O-sulfotransferases (OSTs), to sulfate polysaccharides. Sulfotransferases comprise a family of enzymes that catalyze the transfer of a sulfonate or sulfuryl group (SO₃) from a sulfo donor compound, i.e. an SO₃-donor molecule, to an acceptor molecule. By way of non-limiting example, the sulfo donor compound or SO₃-donor molecule can be the cofactor 3′-phosphoadenosine-5′-phosphosulfate (PAPS). Even though it is more accurate to call these sulfonation reactions, the term sulfation is still widely used. Therefore, the term “sulfation” as used herein refers to a transfer of a sulfonate or sulfuryl group from one molecule to another.

Sulfotransferases mediate sulfation of different classes of substrates such as carbohydrates, oligosaccharides, peptides, proteins, flavonoids, and steroids for a variety of biological functions including signaling and modulation of receptor binding (Bowman et al., (1999) Chem. Biol. 6, R9-R22; and Falany (1997) FASEB J. 11, 1-2). Within the past few years, many new sulfotransferases have been identified and cloned (Aikawa et al., (1999) J. Biol. Chem. 274, 2690; Dooley (1998) Chemico-Biol. Interact 109, 29; Fukuta et al. (1998) Biochim. Biophys. Act. 1399, 57; Habuchi et al., (1998) J. Biol. Chem. 273, 9208; Mazany et al., (1998) Biochim. Biophys. Act 1407, 92; Nastuk et al. (1998) J. Neuroscience 18, 7167; Ong et al., (1998) J. Biol. Chem. 273, 5190; Ouyang et al., (1998) J. Biol. Chem. 273, 24770; Saeki et al. (1998) J. Biochem. 124, 55; Uchimura et al. (1998) J. Biol. Chem. 273, 22577; and Yoshinari et al., (1998) J. Biochem. 123, 740).

As used herein, the term “O-sulfotransferase (OST)” includes polypeptides and nucleic acids encoding HS O-sulfotransferases, such as for example “2-OST” (e.g., mouse 2-OST, GENBANK® Accession No. AAC40135 (SEQ ID NO:1); “3-OST-1” (e.g., human 3-OST-1, GENBANK® Accession No. NP_{—}005105 (SEQ ID NO:2); “3-OST-3” (e.g., human 3-OST-3A, GENBANK®Accession No. NP_{—}006033 (SEQ ID NO:3) and human 3-OST-3B, GENBANK® Accession No. NP_{—}006032 (SEQ ID NO:4); and “6-OST” (e.g., mouse 6-OST-1, GENBANK® Accession No. NP_{—}056633 (SEQ ID NO:5), mouse 6-OST-2, GENBANK® Accession No. BAA89247 (SEQ ID NO:6), and mouse 6-OST-3, GENBANK® Accession No. NP_{—}056635 (SEQ ID NO:7)), which are HS 2-O-sulfotransferase, HS 3-O-sulfotransferase isoform 1, HS 3-O-sulfotransferase isoform 3, and HS 6-O-sulfotransferase, respectively.

The term “OST” includes invertebrate and vertebrate homologs of the O-sulfotransferases (e.g., mammalian (such as human and mouse), insect, and avian homologs). As such, although exemplary embodiments of particular OSTs have been disclosed herein, the presently disclosed subject matter is not intended to be limited to the disclosed examples, but rather “OST”, including particular OSTs (e.g., 2-OST, 3-OST-1,3-OST-3, and 6-OST), includes all comparable OSTs known to the skilled artisan.

The terms “OST gene product”, “OST protein”, and “OST polypeptide” refer to peptides having amino acid sequences which are substantially identical to native amino acid sequences from the organism of interest and which are biologically active in that they comprise all or a part of the amino acid sequence of a HS O-sulfotransferase isoform, or cross-react with antibodies raised against a HS O-sulfotransferase isoform polypeptide, or retain all or some of the biological activity of the native amino acid sequence or protein. Such biological activity can include immunogenicity.

The terms “OST gene product”, “OST protein”, and “OST polypeptide” also include analogs of HS O-sulfotransferase molecules. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct OST analogs. There is no need for a “OST gene product”, “OST protein”, and “OST polypeptide” to comprise all or substantially all of the amino acid sequence of a native OST gene product. Shorter or longer sequences are anticipated to be of use in the presently disclosed subject matter, shorter sequences are herein referred to as “segments.” Thus, the terms “OST gene product”, “OST protein”, and “OST polypeptide” also include fusion or recombinant HS O-sulfotransferase polypeptides and proteins comprising sequences of the OST protein. Methods of preparing such proteins are known in the art.

The terms “OST gene”, “OST gene sequence”, and “OST gene segment” refer to any DNA sequence that is substantially identical to a polynucleotide sequence encoding a HS O-sulfotransferase isoform gene product, protein or polypeptide as defined above, and can also comprise any combination of associated control sequences. The terms also refer to RNA, or antisense sequences, complementary to such DNA sequences. As used herein, the term “DNA segment” refers to a DNA molecule that has been isolated free of total genomic DNA of a particular species. Furthermore, a DNA segment encoding a HS O-sulfotransferase polypeptide refers to a DNA segment that contains OST coding sequences, yet is isolated away from, or purified free from, total genomic DNA of a source species, such as for example Homo sapiens. Included within the term “DNA segment” are DNA segments and smaller fragments of such segments, and also recombinant vectors, including, for example, plasmids, cosmids, phages, viruses, and the like.

The term “substantially identical”, when used to define either a OST gene product or amino acid sequence, or a OST gene or nucleic acid sequence, means that a particular sequence varies from the sequence of a natural OST by one or more deletions, substitutions, or additions, the net effect of which is to retain at least some of biological activity of the natural gene, gene product, or sequence. Such sequences include “mutant” sequences, or sequences in which the biological activity is altered to some degree but retains at least some of the original biological activity.

Alternatively, DNA analog sequences are “substantially identical” to specific DNA sequences disclosed herein if: (a) the DNA analog sequence is derived from coding regions of the natural OST gene; or (b) the DNA analog sequence is capable of hybridization of DNA sequences of (a) under stringent conditions and which encode biologically active OST gene products; or (c) the DNA sequences are degenerate as a result of alternative genetic code to the DNA analog sequences defined in (a) and/or (b). Substantially identical analog proteins will be greater than about 60%, 70%, 80%, 90%, 95%, 96%, 97%, 98%, or 99% identical to the corresponding sequence of the native protein. Sequences having lesser degrees of identity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

Sequence identity or percent similarity of a DNA or peptide sequence can be determined, for example, by comparing sequence information using the GAP computer program, available from the University of Wisconsin Geneticist Computer Group. The GAP program utilizes the alignment method of Needleman et al. (1970) J Mol Biol 48:443, as revised by Smith et al. (1981) Adv Appl Math 2:482. Briefly, the GAP program defines similarity as the number of aligned symbols (i.e., nucleotides or amino acids) that are similar, divided by the total number of symbols in the shorter of the two sequences. The preferred parameters for the GAP program are the default parameters, which do not impose a penalty for end gaps. See Schwartz et al. (1979) Nuc Acids Res 6(2):745-755; Gribskov et al. (1986) Nuc Acids Res 14(1):327-334.

In certain embodiments, the present subject matter concerns the use of OST genes and gene products that include within their respective sequences a sequence that is essentially that of an OST gene, or the corresponding protein. The term “a sequence essentially as that of an OST gene”, means that the sequence is substantially identical or substantially similar to a portion of an OST gene and contains a minority of bases or amino acids (whether DNA or protein) which are not identical to those of an OST protein or an OST gene, or which are not a biologically functional equivalent. The term “biologically functional equivalent” is well understood in the art and is further defined in detail herein. Nucleotide sequences are “essentially the same” where they have between about 75% and about 85% or more preferably, between about 86% and about 90%, or more preferably greater than 90%, or more preferably between about 91% and about 95%, or even more preferably, between about 96% and about 99%; of nucleic acid residues which are identical to the nucleotide sequence of a OST gene. Similarly, peptide sequences which have about 60%, 70%, 80%, or 90%, or preferably from 90-95%, or more preferably greater than 96%, or more preferably 95-98%, or most preferably 96%, 97%, 98%, or 99% amino acids which are identical or functionally equivalent or biologically functionally equivalent to the amino acids of an OST polypeptide will be sequences which are “essentially the same”.

OST gene products and OST encoding nucleic acid sequences, which have functionally equivalent codons, are also covered by the presently disclosed subject matter. The term “functionally equivalent codon” is used herein to refer to codons that encode the same amino acid, such as the ACG and AGU codons for serine. Applicants contemplate substitution of functionally equivalent codons of Table 1 into sequences of OSTs disclosed herein as equivalents.

TABLE 1
Functionally Equivalent Codons

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic Acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | ACG AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood by those of skill in the art that amino acid and nucleic acid sequences can include additional residues, such as additional N- or C-terminal amino acids or 5′ or 3′ nucleic acid sequences, and yet still be encompassed by the OSTs disclosed herein, so long as the sequence retains biological protein activity where protein expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

The present subject matter also encompasses the use of nucleotide segments that are complementary to the sequences of the present subject matter, in one embodiment, segments that are fully complementary, i.e. complementary for their entire length. Nucleic acid sequences that are “complementary” are those, which are base-paired according to the standard Watson-Crick complementarity rules. As used herein, the term “complementary sequences” means nucleic acid sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

One technique in the art for assessing complementary sequences and/or isolating complementary nucleotide sequences is hybridization. Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and preferably in excess of about 45° C. Stringent salt conditions will ordinarily be less than about 1,000 mM, typically less than about 500 mM, and preferably less than about 200 mM. However, the combination of parameters is much more important than the measure of any single parameter. See e.g., Wethmur & Davidson (1968) J Mol Biol 31:349-370. Determining appropriate hybridization conditions to identify and/or isolate sequences containing high levels of homology is well known in the art. See e.g., Sambrook et al. (2001) Molecular Cloning: A Laboratory Manual, Third Edition, Cold Spring Harbor Laboratory Press, Cold Spring Harbor, N.Y.

For the purposes of specifying conditions of high stringency, preferred conditions are salt concentration of about 200 mM and temperature of about 45° C. One example of such stringent conditions is hybridization at 4×SSC, at 65° C., followed by a washing in 0.1×SSC at 65° C. for one hour. Another exemplary stringent hybridization scheme uses 50% formamide, 4×SSC at 42° C. Another example of “stringent conditions” refers to conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. Nucleic acids having sequence similarity are detected by hybridization under low stringency conditions, for example, at 50° C. and 10×SSC (0.9 M NaCl/0.09 M sodium citrate) and remain bound when subjected to washing at 55° C. in 1×SSC. Sequence identity can be determined by hybridization under stringent conditions, for example, at 50° C. or higher and 0.1×SSC (9 mM NaCl/0.9 mM sodium citrate).

Nucleic acids that are substantially identical to the provided OSTs, e.g., allelic variants, genetically altered versions of the gene, etc., bind to the disclosed OSTs under stringent hybridization conditions. By using probes, particularly labeled probes of DNA sequences, one can isolate homologous or related genes. The source of homologous genes can be any species, e.g., primate species, particularly human; rodents, such as rats and mice; canines; felines; bovines; ovines; equines; insects; yeasts; nematodes; etc.

Between mammalian species, e.g., human, mouse and rat, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence will usually be at least about 18 nucleotides long, more usually at least about 30 nucleotides long, and can extend to the complete sequence that is being compared. Algorithms for sequence analysis are known in the art, such as BLAST, described in Altschul et al. (1990) J Mol Biol 215:403-410. The sequences provided herein are essential for recognizing OST related and homologous proteins in database searches.

At a biological level, identity is just that, i.e. the same amino acid at the same relative position in a given family member of a gene family. Homology and similarity are generally viewed as broader terms. For example, biochemically similar amino acids, for example leucine and isoleucine or glutamate/aspartate, can be present at the same position—these are not identical per se, but are biochemically “similar”. As disclosed herein, these are referred to as conservative differences or conservative substitutions. This differs from a conservative mutation at the DNA level, which changes the nucleotide sequence without making a change in the encoded amino acid, e.g., TCC to TCA, both of which encode serine.

When percentages are referred to herein, it is meant to refer to percent identity. The percent identities referenced herein can be generated by alignments with the program GENEWORKS™ (Oxford Molecular, Inc. of Campbell, Calif., U.S.A.) and/or the BLAST program at the NCBI website. Another commonly used alignment program is entitled CLUSTAL W and is described in Thompson et al. (1994) Nucleic Acids Res 22(22):4673-4680, among other places.

The term “gene” is used for simplicity to refer to a functional protein, polypeptide or peptide encoding unit. As will be understood by those in the art, this functional term includes both genomic sequences and cDNA sequences.

As noted above, modifications and changes can be made in the structure of the OST proteins and peptides described herein and still constitute a molecule having like or otherwise desirable characteristics. For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the native protein, but this need not be the case, and the biological activity of the presently disclosed subject matter is not limited to a particular mechanism of action. It is thus contemplated in accordance with the present subject matter that various changes can be made in the sequence of the OST proteins and peptides or underlying nucleic acid sequence without appreciable loss of their biological utility or activity.

Biologically functional equivalent peptides, as used herein, are peptides in which certain, but not most or all, of the amino acids can be substituted. Thus, applicants contemplate substitution of codons that encode biologically equivalent amino acids as described herein into the sequences of the disclosed OSTs, but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged. Changes designed by man can be introduced through the application of site-directed mutagenesis techniques, e.g., to introduce improvements to the antigenicity of the protein or to test OST mutants in order to examine OST sulfotransferase activity, or other activity at the molecular level.

Amino acid substitutions, such as those which might be employed in modifying the OST proteins and peptides described herein, are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of skill in the art.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al. (1982) Mol Biol 157:105, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those, which are within ±1 of the original value, are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those, which are within ±1 of the original value, are particularly preferred, and those within ±0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that the presently disclosed subject matter is not limited to the particular nucleic acid and amino acid sequences of the OSTs disclosed herein. Recombinant vectors and isolated DNA segments can therefore variously include the O-sulfotransferase polypeptide-encoding region itself, include coding regions bearing selected alterations or modifications in the basic coding region, or include larger polypeptides which nevertheless comprise the O-sulfotransferase polypeptide-encoding regions or can encode biologically functional equivalent proteins or peptides which have variant amino acid sequences. Biological activity of an O-sulfotransferase can be determined using techniques generally known in the art, for example as disclosed herein in the Examples.

The nucleic acid segments of the present subject matter, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in any of the OSTs disclosed herein, such as about 10 nucleotides, and which are up to 10,000 or 5,000 base pairs in length, with segments of 3,000 being preferred in certain cases. DNA segments with total lengths of about 4,000, 3,000, 2,000, 1,000, 500, 200, 100, and about 50 base pairs in length are also contemplated to be useful.

Recombinant vectors form further aspects of the present subject matter. Particularly useful vectors are those in which the coding portion of the DNA segment is positioned under the control of a promoter. The promoter can be that naturally associated with the OST gene, as can be obtained by isolating the 5′ non-coding sequences located upstream of the coding segment or exon, for example, using recombinant cloning and/or polymerase chain reaction (PCR) technology and/or other methods known in the art, in conjunction with the compositions disclosed herein.

In other embodiments, it is provided that certain advantages will be gained by positioning the coding DNA segment under the control of a recombinant, or heterologous, promoter. As used herein, a recombinant or heterologous promoter is a promoter that is not normally associated with a 3-O-sulfotransferase gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (See, e.g., Sambrook et al. (2001) Molecular Cloning: A Laboratory Manual, Third Edition, Cold Spring Harbor Laboratory Press, Cold Spring Harbor, N.Y.). The promoters employed can be constitutive or inducible and can be used under the appropriate conditions to direct high level expression of the introduced DNA segment, such as is advantageous in the large-scale production of recombinant proteins or peptides.

In some embodiments of the method disclosed herein for sulfating polysaccharides, the OST enzyme is immobilized on a substrate. This provides an advantage in that the substrate to which the OSTs are attached can be washed after a sulfation reaction to remove all components of the reaction except the bound OSTs. As such, the products of the reaction can be more easily separated from the enzymes catalyzing the reaction and the OSTs can be recycled and utilized again in multiple sulfation reactions. In some embodiments, the substrate is agarose. In particular embodiments, the agarose substrate is an agarose bead and the OSTs are linked to the beads.

II.B. Reduction of Inhibitory Effects of PAP

The presently disclosed method for sulfating polysaccharides can comprise providing a “reaction condition” that modifies PAP to reduce inhibitory effects of PAP, such as competing with PAPS for binding with OSTs, on the polysaccharide sulfation. In some embodiments, the reaction condition comprises a phosphatase enzyme. The phosphatase enzyme can remove a phosphate from the PAP, which reduces its binding affinity for OSTs. In some embodiments, the phosphatase is 3′-ribonucleotide phosphohydrolase.

In some embodiments, the reaction condition is a PAPS regeneration system, which comprises a PAPS regenerating enzyme and a sulfo donor compound. The PAPS regenerating enzyme catalyzes regeneration of the PAPS from the PAP utilizing the sulfo donor compound as a substrate. See, e.g., U.S. Pat. No. 6,255,088; and Burkart et al., (2000) J. Org. Chem. 65, 5565-5574, both of which are herein incorporated by reference in their entirety. Thus, the PAPS regeneration system provides the dual advantages of reducing the inhibitory effects of PAP accumulation on sulfotransferase activity while also constantly “recharging” the reaction mixture with the primary sulfo donor molecule, PAPS. In some embodiments, the PAPS regenerating enzyme is an estrogen sulfotransferase.

Thus, an aspect of the presently disclosed subject matter is directed to a sulfo donor compound (e.g., PAPS) regeneration process coupled with sulfation of a polysaccharide substrate. In particular, the process can be of a type wherein the sulfation of a polysaccharide substrate is catalyzed by a sulfotransferase, such as one or more OSTs, with a conversion of 3′-phosphoadenosine-5′-phosphosulfate (PAPS) to adenosine 3′,5′-diphosphate (PAP). The sulfation process can be coupled with an enzymatic regeneration of the PAPS from the PAP. The enzymatic regeneration can employ an arylsulfotransferase as the catalyst and an aryl sulfate as a substrate. In some embodiments, the enzymatic regeneration can comprise a human or mouse estrogen sulfotransferase (EST). As previously disclosed, preferred carbohydrate substrates can include GAGs, such as for example heparan sulfates, including heparin.

In some embodiments, the arylsulfotransferase is a recombinant aryl sulfotransferase IV (AST-IV; e.g., rat AST-IV (SEQ ID NO:8)). This enzyme, when coupled to a sulfotransferase of choice, transfers sulfate from an aryl sulfate (e.g., p-nitrophenyl sulfate (PNPS) or any similar compound containing a phenol sulfate moiety) to PAP. This system averts product inhibition by PAP while regenerating PAPS in situ and can be monitored quantitatively by measurement of the absorbance of released p-nitrophenol at 400 nm. In some embodiments, human and/or mouse estrogen sulfotransferase (EST) can be used in place of or in conjunction with AST-IV.

The enzyme AST-IV exists in two oxidative forms (Marshall et al., (1997) J. Biol. Chem. 272, 9153-9160; Marshall et al., (1998) Chem.-Biol. Interact 109, 107-116; Yang et al., (1998) Chem.-Biol. Interact 109, 129-135; Yang et al. (1996) Protein Expression Purif. 8, 423-429; Guo et al. (1994) Chem.-Biol. Interact. 92, 25-31; Chen et al. (1992) Protein Expression Purif. 3, 421-6; Lin et al. (1998) Anal. Biochem. 264, 111-117; and Yang et al., (1997) Protein Eng. 10, 70). These two oxidative forms can be easily resolved (Yang et al. (1996) Protein Expression Purif. 8, 423-429), and the resolved physiologically relevant form has been utilized to assay picomole quantities of PAPS and PAP (Lin et al. (1998) Anal. Biochem. 264, 111-117). As the bacterial expression of rat AST-IV has been demonstrated (Chen et al., (1992) Protein Expression Purif. 3, 421-6; and Ozawa et al., (1990) Nucleic Acids Res. 18, 4001z.), AST IV can be cloned from a rat liver cDNA library, overexpressed in a recombinant bacterial system (e.g., E. coli) and isolated (See, e.g., U.S. Pat. No. 6,255,088, herein incorporated by reference in its entirety).

Coupling the sulfotransferase catalyzed sulfation reaction with a PAPS regeneration system can provide a further advantage of generating PAPS utilized in the reaction directly from PAP. That is, the reaction mixture can be formulated to combine PAP with a PAPS regenerating enzyme prior to or simultaneously with addition of a sulfotransferase to the reaction mixture. The PAPS regenerating enzyme can then generate PAPS from the PAP for use by the sulfotransferase, thereby alleviating the need of supplying any of the more expensive and unstable PAPS to the reaction mixture. For example, coupling the PAPS regeneration system to use PNPS as a sulfo donor can potentially reduce the cost of the synthesis of sulfated polysaccharides by as much as 1,000-fold. As such, in some embodiments of the presently disclosed subject matter a method of sulfating a polysaccharide is provided comprising providing a reaction mixture comprising therein adenosine 3′,5′-diphosphate (PAP), a PAPS regenerating enzyme and a sulfo donor compound (other than PAPS, e.g., PNPS) and incubating the reaction mixture for a time period sufficient to catalyze the production of 3′-phosphoadenosine 5′-phosphosulfate (PAPS) from the PAP by the PAPS regenerating enzyme utilizing the sulfo donor compound as a substrate. The method further comprises incubating a polysaccharide substrate and at least one O-sulfotransferase (OST) enzyme with the reaction mixture, wherein production of a sulfated polysaccharide from the polysaccharide substrate is catalyzed by the OST enzyme with a conversion of the PAPS to PAP and wherein the PAPS regenerating enzyme then catalyzes regeneration of the PAPS from the PAP, again utilizing the sulfo donor compound as a substrate.

II.C. Synthesizing Iduronic Acid-Free HS

As noted herein, the presently disclosed subject matter can be adapted to produce a multitude of sulfated polysaccharides, i.e. HS-like compounds, having pre-selected biological activities using an enzyme-based combinatorial approach. In essence, the process of reacting a polysaccharide substrate with specified OSTs can be continued until a final polysaccharide is produced exhibiting the desired biological activities, wherein the activities are based, at least in part, on the sulfation pattern of the polysaccharide.

The presently disclosed subject matter also illustrates that the presence or absence of an iduronic acid (IdoUA) residue can also impart biological properties on the HS-like compound. In particular, the methods described herein provide for the synthesis of HS-like compounds without IdoUA residues, i.e. IdoUA-free HS-like compounds. By omitting the inclusion of epimerase in the synthesis of HS-like compounds, the epimerization of the glucuronic acid residue to an iduronic acid residue is precluded, which results in the synthesis of a HS-like compound that is substantially free of IdoUA. As described in more detail in the Examples below, the synthesis of IdoUA-free HS-like compounds can provide for the synthesis of compounds with the desired biological properties of HS without untoward biological effects. In some embodiments, the HS-like compounds described herein are isolated and/or purified.

In some embodiments, the terms “IdoUA-free” and “iduronic acid-free” refer to the substantial absence of iduronic acid. In some embodiments, the terms “IdoUA-free HS” and “IdoUA-free HS-like compound” refer to HS-like compounds substantially devoid of iduronic acid. In some embodiments, IdoUA-free and iduronic acid-free can refer to 0, 0.1, 0.5, 1, 2, 3, 4 or 5% iduronic acid. In some embodiments, IdoUA-free can refer to an amount of IdoUA below limits of detection using 1H-NMR.

In some embodiments, an IdoUA-free HS-like compound can be synthesized by incubating a polysaccharide substrate (e.g., N-sulfo heparosan) with 2-OST, 6-OST-1,6-OST-3 and 3-OST-1 (compound 1, Table 2), wherein the reaction is substantially free of epimerase. In another embodiment, a polysaccharide substrate is incubated with 6-OST-1,6-OST-3 and 3-OST-1 to produce compound 2, which also has no IdoUA residue due to the substantial absence of epimerase. In still yet another embodiment, an IdoUA-free HS-like compound can be synthesized by reacting a polysaccharide substrate with 3-OST-1 and 2-OST with substantially no epimerase (compound 3). Although exemplary embodiments of particular HS-like compounds or molecules have been disclosed herein, the presently disclosed subject matter is not intended to be limited to the disclosed examples, but rather HS-like compounds include all comparable synthetically sulfated polysaccharides known to one of ordinary skill in the art. In particular, it is envisioned that one of ordinary skill in the art, upon review of the instant disclosure, will be capable of producing numerous HS-like compounds based upon the disclosed methods.

In some embodiments, the terms “substantially free”, “substantial absence” and “substantially no” refer to a compound or entity that is predominantly devoid or lacking. In some embodiments, these terms refer to a compound or entity that is entirely devoid or lacking. By way of non-limiting example, substantially free, substantial absence and substantially no can refer to the presence of 0, 0.1, 0.5, 1, 2, 3, 4 or 5% of a compound or entity, and/or to the presence of an amount of compound or entity below the limits of a standard detection approach.

In some embodiments, the terms “substantially free of epimerase”, “substantial absence of epimerase” and “substantially no epimerase” refer to reactions for the synthesis of HS-like compounds, wherein the reactions are predominantly devoid of or lacking in epimerase. In some embodiments, substantially free of epimerase, substantial absence of epimerase and substantially no epimerase can refer to 0, 0.1, 0.5, 1, 2, 3, 4 or 5% epimerase, and/or to the presence of an amount of epimerase below the limits of a standard detection approach.

### III. Methods of Determining the Mechanism of Activity of HS-Like Compounds

The presently disclosed subject matter provides methods for determining a mechanism of activity of HS-like compounds. The enzyme-based combinatorial approach of synthesizing HS-like compounds provides for the systematic manipulation of the structural composition of HS-like compounds. For example, the presently disclosed subject matter provides for the synthesis of HS-like compounds with specific sulfation patterns. FIG. 1B schematically illustrates the enzyme-based combinatorial approach to synthesizing HS-like compounds by selectively reacting a polysaccharide substrate with different OSTs and biosynthetic enzymes (Table 2). As such, this method provides a model system to better understand the biosynthesis of HS-like compounds as well as the functional characteristics of each of the components that make up HS-like compounds.

In some embodiments, the presently disclosed subject matter provides a method of determining the mechanism of activity of HS-like compounds, the method comprising, synthesizing one or more HS-like compounds by incubating one or more polysaccharide substrates with a sulfo donor compound and one or more combinations of biosynthetic enzymes, the biosynthetic enzymes comprising epimerases, N-sulfotransferases and O-sulfotransferases; subjecting one or more of the HS-like compounds to a test for an activity; and determining a mechanism of activity of the HS-like compounds based on one or more results of the one or more HS-like compounds. The method can further comprise providing a reaction mixture comprising adenosine 3′,5′-diphosphate (PAP), a PAPS regenerating enzyme and a sulfo donor compound; and incubating the reaction mixture for a time period sufficient to catalyze the production of 3′-phosphoadenosine 5′-phosphosulfate (PAPS), whereby the sulfo donor compound is provided.

In some embodiments the test used to elucidate a mechanism of a component of a HS-like compound can comprise an anti-thrombin binding activity test. For example, two or more HS-like compounds, each synthesized using different enzyme-based combinatorial reaction schemes and thereby differing in their structural composition, can be analyzed for anti-thrombin binding activity via an anti-thrombin binding activity test, to thereby elucidate the one or more structural components of the HS-like compounds responsible for or relevant to the anti-thrombin activity. Likewise, in other embodiments, the test used to elucidate a mechanism of a component of a HS-like compound can comprise anti-Xa activity, anti-IIa activity, cell proliferation stimulation activity or cell growth stimulation activity. Still yet, in other embodiments, the test used to elucidate a mechanism of a component of a HS-like compound can comprise activated partial thromboplastin time (APTT) or prothrombin time (PT).

In some embodiments, the disclosed method of determining the mechanism of activity of HS-like compounds comprises synthesizing one or more HS-like compounds chemically. Still yet, in some embodiments the disclosed method of determining the mechanism of activity of HS-like compounds comprises synthesizing one or more HS-like compounds using a combination of enzymatic and chemical methods.

### IV. Kits

The presently disclosed subject matter further provides kits for synthesizing HS-like compounds In some embodiments, the kit comprises at least one sulfotransferase enzyme (e.g., at least one OST); and a reagent which modifies an SO₃-donor, e.g., adenosine 3′,5′-diphosphate (PAP), to reduce an inhibitory effect of the SO₃-donor on the polysaccharide sulfation. In some embodiments of the kit the at least one sulfotransferase enzyme is contained within a first container and the reagent is contained within a second container. The kit can further comprise instructions for sulfating a polysaccharide.

In some embodiments of the kit, the at least one sulfotransferase enzyme is an OST enzyme selected from the group consisting of 2-OST, 3-OST-1,3-OST-3, 6-OST, and combinations thereof. In some embodiments, the OST enzyme is a recombinant OST enzyme, such as for example a recombinant OST enzyme produced in a bacterial expression system. In some embodiments, the OST enzyme is a fusion protein. Further, in some embodiments, the OST enzyme is immobilized to a substrate, such as for example an agarose bead.

In some embodiments of the kit, the reagent comprises a PAPS regeneration system comprising a PAPS regenerating enzyme (e.g., AST-IV) and a sulfo donor compound (e.g., PNPS). In other embodiments of the kit, the reagent comprises a phosphatase enzyme (e.g., 3′-ribonucleotide phosphohydrolase). In some embodiments, human and/or mouse EST can be provided in place of or in conjunction with AST-IV.

In some embodiments, the kit provides for the synthesis of isolated and/or purified HS-like compounds described herein.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Materials and Methods for Examples 1-5

Expression of HS biosynthetic enzymes. Expressions of HS biosynthetic enzymes, including NST, epimerase, 2-OST, 6-OST-1 and 3-OST-1, were carried out in E. coli as described previously (Chen et al. (2005) Chemistry and Biology 12, 731-756; Kakuta et al. (2003) Biochem. Soc. Trans. 31, 331-334; Edavettal et al. (2004) J. Biol. Chem. 279, 25789-25797; Munoz et al. (2006) Biochem. 45, 5122-5128). The expression of 6-OST-3 was also carried out in E. coli. The catalytic domain of mouse 6-OST-3 (Pro¹²¹-Pro⁴⁵⁰) was cloned into a pMalc2x vector (New England BioLab) from mouse brain Quick clone cDNA library (BD Biosciences). The expression was carried out in Origami-B cells (Novagen) carrying pGro7 (Takara, Japan) plasmid expressing chaperonin proteins GroEL and GroES of E. coli. Transformed cells were grown in 6 liters of LB medium supplemented with 2 mg/ml glucose, 12.5 μg/ml tetracycline, 15 μg/ml kanamycin, 35 μg/ml chloramphenicol, and 50 μg/ml carbenicillin at 37° C. When the A₆₀₀ reached 0.4-0.7, isopropyl-thiogalactopyranoside (0.15 mM) and L-arabinose (1 mg/ml) were added to induce the expression of 6-OST-3 and chaperonin proteins, respectively. The cells were allowed to shake overnight at 22° C. Purification was carried out using an amyloseagarose (New England BioLab) column following the protocols provided by the manufacturer.

Preparation of enzymatically modified polysaccharides. N-Sulfo Heparosan was prepared by incubating chemically deacetylated heparosan with purified NST and the components for PAPS regeneration system as described below. N-sulfo heparosan (150 μg) was incubated with [³⁵S]PAPS (120 μM, 4.5×10³ cpm/nmole) and the combination of epimerase (2.7 mg if included), 2-OST (8 mg if included), 6-OST-1 (1.2 mg if included), 6-OST-3 (1.4 mg if included) and 3-OST-1 (0.9 mg if included) in 50 mM MES and 1% Triton X-100, pH 7.0. The reactions were incubated at 37° C. for 2 hrs with shaking. The resultant ³⁵S-labeled polysaccharides were purified by DEAE chromatography (Chen et al. (2005) Chemistry and Biology 12, 731-756).

To prepare the enzymatically modified polysaccharides, a polysaccharide substrate, in some embodiments an N-[³⁵S]sulfo oligosaccharide substrate (1 μg, 1,500 cpm), was incubated with a mixture of OST enzymes, in some embodiments 6-OST-1 (20 μg), 6-OST-3 (20 μg) and 3-OST-1 (10 μg), and [³⁵S]PAPS (50 μM, 2.85×10⁵ cpm/nmole) in 100 μl of 50 mM MES and 1% Triton X-100. Alternatively, a polysaccharide substrate (1 μg) was incubated with epimerase (7 μg) at 37° C. for 30 min before it was incubated with a mixture of OST enzymes, in some embodiments 2-OST (40 μg), 6-OST-1 (20 μg), 6-OST-3 (20 μg) and 3-OST-1 (10 μg). The reactions were terminated by heating at 100° C. for 2 min. The resultant sulfated polysaccharides were mixed with AT to determine the bindings using a ConA-agarose column as described below.

Disaccharide analysis of polysaccharides. The ³⁵S-labeled HS was degraded with nitrous acid at pH 1.5, followed by reduction with sodium borohydride (Shively et al. (1976) Biochem. 15, 3932-3942). The resultant ³⁵S-labeled disaccharides were resolved by a C₁₈ reversed phase column (0.46×25 cm) (Vydac) under reversed phase ion pairing (RPIP)HPLC conditions. The identities of the disaccharides were determined by coeluting with appropriate ³⁵S-labeled disaccharide standards (Chen et al. (2003) Glycobiology 13, 785-794).

Determination of the amount of polysaccharides. The amount of the synthetic polysaccharides was estimated by a colorimetric method using alcian as described (Bjornsson, S. (1993) Anal. Biochem. 210, 282-291), where the standard curve was generated using HS isolated from bovine kidney. To measure the concentration of heparosan, N-sulfo heparosan, and N-sulfo heparosan 6-O-sulfate, the polysaccharides were depolymerized to disaccharides by a mixture of heparin lyase I, II and III. The amount of the resultant disaccharides was determined by coeluting with disaccharide standards (from Seikagaku) on RPIP-HPLC as described above using UV 232 nm detection.

Antithrombin-binding. Approximately 1×05 cpm of [³⁵S]-labeled compound was incubated with 5 μg of human AT (Cutter Biological) in 50 μl binding buffer containing 10 mM Tris-HCl, pH 7.5, 150 mM NaCl, 1 mM Mn²⁺, 1 mM Mg²⁺, 1 mM Ca²⁺, 10 μM dextran sulfate, 0.0004% Triton X-100, and 0.02% sodium azide for 30 min at room temperature. Concanavalin A (ConA)-Sepharose (Sigma, 50 μl of 1:1 slurry) was then added and the reaction was shaken at room temperature for 1 h. The beads were then washed by 3×1 ml binding buffer, and the bound polysaccharide was eluted with 1 M NaCl.

Anti-Xa and anti-IIa assays. Assays were based on two previous methods (Zhang et al. (2001) J. Biol. Chem. 276, 42311-42321; Duncan et al. (2004) Biochim Biophys Acta 1671, 34-43). Briefly, factor Xa (Enzyme Research Laboratories, South Bend, Ind.) and thrombin (Sigma) were diluted to 1 unit/ml and 8 units/ml with PBS containing 1 mg/ml BSA, respectively. AT was diluted with PBS containing 1 mg/ml BSA to give a stock solution at the concentration of 27 μM. The chromogenic substrates, S-2765 (for factor Xa assay) and S-2238 (for thrombin assay), were from Diapharma and prepared at 1 mM with PBS. The synthesized polysaccharides or heparin was dissolved in a buffer containing 50 mM Tris-HCl, pH 8.4, 7.5 mM Na₂EDTA, 175 mM NaCl at various concentrations (1 ng/ml to 10,000 ng/ml). The reaction mixture, which consisted of 25 μl of AT stock solution and 25 μl of the solution containing polysaccharide, was incubated at 37° C. for 2 min. Factor Xa (25 μl) or thrombin (25 μl) was added. After incubating 37° C. for 4 min, 25 μl of S-2765 or S-2238 was added. The absorbance of the reaction mixture was measured at 405 nm continuously for 10 min. The absorbance values were plotted against the reaction time. The initial reaction rates as a function of concentration were used to calculate the IC₅₀ values.

Affinity coelectrophoresis. The dissociation constant (K_{d}) of each sample and AT was determined using affinity co-electrophoresis (Lee et al. (1991) Proc. Natl. Acad. Sci. USA 88, 2768-2772). Approximately 4000-5000 cpm of antithrombin-binding ³⁵S-labeled polysaccharide was loaded per lane with zones of AT at concentrations 0, 8, 16, 60 and 120 nM. The gel was run at 400 mA for 2 hours, dried and then analyzed on a PhosphoImager (Amersham Biosciences, Storm 860). The retardation coefficient was calculated at R=(M₀−M)/M, where M₀ is the mobility of the polysaccharide through the zone without AT, and M is the mobility of the polysaccharide through each separation zone. The retardation coefficient was then plotted against the retardation coefficient divided by its respective concentration of AT. The slope of the line represents −1/K_{d}.

FGF2/FGFR1C-mediated proliferation assay. BaF3 cells ectopically expressing FGFR1C have been previously described (Ornitz et al. (1996) J. Biol. Chem. 271, 15292-15297). The BaF3-FGFR1c cells were maintained in RPMI 1640 media (Sigma, St. Louis, Mo.) supplemented with 10% fetal bovine serum, 0.5 ng/ml IL-3 (PeproTech Inc., Rocky Hill, N.J.), 2 mM L-glutamine, penicillin (50 IU/ml) and streptomycin (50 μg/ml), and 50 μM β-mercaptoethanol. For mitogenic assays, BaF3 FGFR1c cells were washed three times with RPMI 1640 media to remove IL-3 and resuspended in the growth media lacking IL-3. About 30,000 cells were plated per well in a 96-well plate in media containing various concentrations of heparin, compounds 1, 2, 7, and 8 and 2 nM of FGF-2 (PeproTech) in a total volume of 200 μl. The cells were then incubated at 37° C. for 40 h. To each well, an additional 50 μl of growth media containing 1 μCi of [³H]thymidine was added. Cells were harvested after 4-5 h by centrifugation. The incorporation of [³H]thymidine into the DNA was determined by scintillation counting.

PAPS regeneration system. N-terminal (His)₆ tagged AST-IV was expressed in E. coli and purified as described by Burkart and colleagues (Burkart et al. (2000) J. Org. Chem. 65, 5565-5574) at a yield≈50 mg/liter of bacterial culture.

Preparation of N-sulfo heparosan. Heparosan was isolated from E. coli K5 strain using a DEAE column (Vann et al. (1981) Eur. J. Biochem. 116, 359-364). The deacetylated hepraosan was achieved under alkaline conditions (Lindahl et al. (2005) J. Med. Chem. 48, 349-352). The deacetylated heparosan was incubated with purified NST and coupled with a PAPS regeneration system. Briefly, 2.5 mg purified AST-IV was incubated with 40 μM PAP and 1 mM p-nitrophenol sulfate (PNPS) in 40 ml of 50 mM MES, pH 7.0, 1% Triton X-100, 1 mM MgCl₂, and 1 mM MnCl₂ at 25° C. for 15 min. The reaction mixture was mixed with N-sulfotransferase (10 mg), 5 mg of deacetylated heparosan added and rotated at 25° C. for 24 h. The N-sulfo heparosan was recovered by a DEAE column. The resultant product was subjected to a mixture of heparin lyases digestion followed by a disaccharide analysis to assess the level of N-sulfation. After two rounds of the modifications, the N-sulfation level reached to 80%.

Preparation of N-sulfo oligosaccharides. N-[³⁵S]sulfo heparosan (1 mg, 1.5×10⁶ cpm) was mixed with 20 ng of purified heparin lyase III in 1 ml of 50 mM sodium phosphate, pH 7.0. The digestion lasted for 18 hrs and was terminated by heating at 100° C. for 2 min. The digested polysaccharides were resolved on a BIO-GEL® P-10 which was eluted with a buffer containing 25 mM Tris and 1000 mM NaCl, pH 7.4 at a flow rate of 2 ml/h.

### Example 1

### Development of an Enzyme-Based Combinatorial Approach to Synthesize HS-Like Compounds with Unique Sulfation Patterns

Biosynthesis of HS. The biosynthesis of HS involves a series of specialized sulfotransferases and other enzymes. Control of the sulfation pattern mainly depends on the substrate specificities of the enzymes involved in the biosynthetic pathway. HS is initially synthesized as a linear copolymer of glucuronic acid (GlcUA) and N-acetylated glucosamine (GlcNAc), which then undergoes various modifications. These modifications are carried out by a series of biosynthetic enzymes, including glucosaminyl N-deacetylase/N-sulfotransferase, which converts the GlcNAc unit to an N-sulfo glucosamine (GlcNS) unit (FIG. 1A). After the N-sulfation, C₅-epimerase converts GlcUA unit to an iduronic acid (IdoUA) unit. The resultant polysaccharide is further modified by 2-O-sulfotransferase (2-OST), 6-O-sulfotransferase (6-OST) and 3-O-sulfotransferase (3-OST) to introduce the 2-O-sulfo group to IdoUA/GlcUA, and the 6-O-sulfo/3-O-sulfo groups to the glucosamine unit, respectively (FIG. 1A). 6-OST is present in three isoforms, and 3-OST is present in seven isoforms.

The presently disclosed subject matter provides methods for synthesizing HS-like compounds with unique sulfation patterns by utilizing an enzyme-based combinatorial approach. By selectively combining collections of HS biosynthetic enzymes the methods disclosed herein provide for the synthesis of a library of HS-like compounds having different sulfation patterns (FIG. 1B; Formula 1). Further, this enzyme-based combinatorial approach can be used as a general method for discovering new HS-like compounds with unique biological functions. Additionally, by systematically altering the sulfation pattern the function of specific sulfo groups can be elucidated.

In some embodiments, the disclosed method of synthesizing of a library of HS-like compounds comprises synthesizing one or more HS-like compounds chemically. Still yet, in some embodiments the disclosed method of synthesizing of a library of HS-like compounds comprises synthesizing one or more HS-like compounds using a combination of enzymatic and chemical methods.

Expression of HS Sulfotransferases in E. coli. The enzymes found in synthesizing biologically active HS-like compounds, including NST, epimerase, and numerous OST enzymes, were expressed and purified as described herein. Four OST enzymes, including 2-OST, 3-OST-1,6-OST-1 and 6-OST-3, were utilized for the synthesis of HS-like compounds. Bacterial expressed 3-OST-1 and 3-OST-3 can exhibit substrate specificity and specific enzymatic activity comparable with those of their counterparts expressed in insect cells (Moon et al. (2004) J. Biol. Chem. 279, 45185-45193; Edavettal et al. (2004) J. Biol. Chem. 279, 25789-25797). Expression of the catalytic domains of 2-OST and 6-OST was also achieved in relatively high yield by preparing a fusion protein with MBP and 2-OST or 6-OST in Rosetta-gami B cells. Because 2-OST and 6-OST fusion proteins were enzymatically active and highly soluble, the MBP domain was retained.

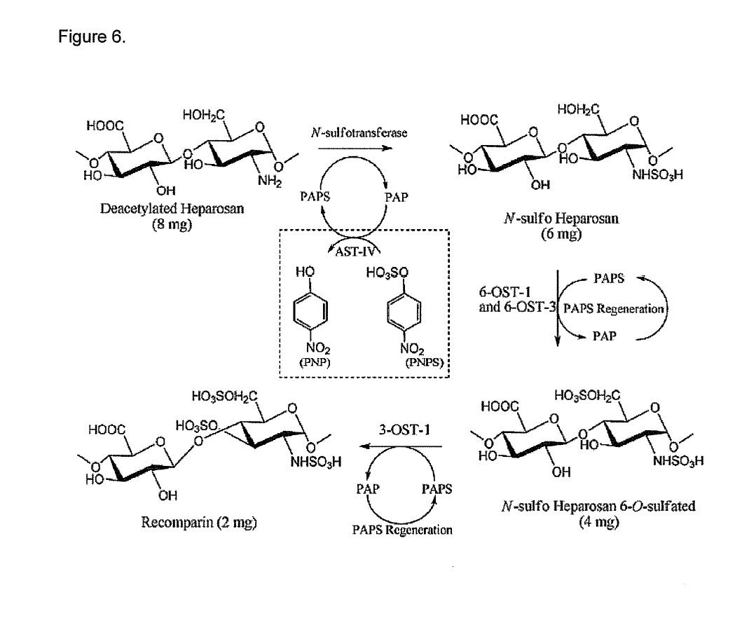

Introduction of PAPS Regeneration System. PAP inhibits sulfotransferase-catalyzed reactions. A PAPS regeneration system can be used to convert PAP to PAPS by relying on AST-IV to catalyze the transfer of the sulfo group from PNPS to PAP, as illustrated in FIG. 6. The presently disclosed subject matter provides for the use of the PAPS regeneration system coupled with O-sulfotransferases. Complete modification of the substrate could be demonstrated by the low susceptibility of polysaccharide product to undergo additional sulfation using [³⁵S]PAPS with soluble enzymes (Chen et al. (2005) J. Biol. Chem. 280, 42817-42825). Under the standard conditions, 2-OST, 6-OST, and 3-OST-1 afforded 98, 97, and 98% complete modification, respectively, using the PAPS regeneration system (Chen et al. (2005) J. Biol. Chem. 280, 42817-42825).

HS Biosynthetic Enzyme Schemes. The presently disclosed subject matter demonstrates that HS-like compounds having specific biological activities can be synthesized by subjecting a backbone saccharide polymer to different enzymatic modifications. N-sulfo heparosan was used as a starting material to prepare HS-like compounds with various O-sulfation patterns (FIG. 1B). In some embodiments, N-sulfation is important for subsequent O-sulfations and/or epimerization. Eight unique HS-like compounds (compounds 1-8) were prepared using the enzyme-based combinatorial approach, wherein eight different combinations of enzymes were designed. The enzyme combinations and compounds are summarized in Table 2. Each of compounds 1-8 comprise a different sulfation pattern, dictated by the particular OSTs used, e.g., 2-OST, 3-OST and 6-OST, as well as the presence or absence of epimerase which converts glucuronic acid to iduronic acid (IdoUA). For example, compound 2 was synthesized by incubating N-[³⁵S]sulfo oligosaccharide substrates, e.g. N-heparosan, (1 μg, 1,500 cpm) with a mixture of 6-OST-1 (20 μg), 6-OST-3 (20 μg) and 3-OST-1 (10 μg) and [³⁵S]PAPS (50 μM, 2.85×10⁵ cpm/nmole) in 100 μl of 50 mM MES and 1% Triton X-100. Alternatively, for the preparation of compound 8, the oligosaccharide substrates (1 μg) were incubated with epimerase (7 μg) at 37° C. for 30 min before incubation with a mixture of 2-OST (40 μg), 6-OST-1 (20 μg), 6-OST-3 (20 μg) and 3-OST-1 (10 μg). The reactions were terminated by heating at 100° C. for 2 min.

TABLE 2
Summary of the synthetic polysaccharides and the results of their bindings to antithrombin (AT)

| | | | O-[³⁵S]sulfation level | Binding to | Binding constant to |
|---|---|---|---|---|---|
| | | | (nmoles/μg | antithrombin affinity | antithrombin (Kd) |
| Compound | Modification enzymes | Structural features¹ | polysaccharide) | column (%)² | (nM)³ |
| 1 | 2-OST, 6-OST-1, | No iduronic acid | 1.4 | 51% | 27 nM |
| | 6-OST-3 and 3-OST-1 | residue | | | |
| 2 | 6-OST-1, 6-OST-3 and | No iduronic acid, | 1.1 | 52% | 57 nM |
| | 3-OST-1 | no 2-O-sulfation | | | |
| 3 | 3-OST-1 and 2-OST | No iduronic acid, | 0.9 | 1.0% | Not determined |
| | | no 6-O-sulfation | | | |
| 4 | Epi⁴, 2-OST and | No 6-O-sulfation | 1.3 | 0.7% | Not determined |
| | 3-OST-1 | | | | |
| 5 | Epi, 2-OST, 6-OST-1 | No 3-O-sulfation | 1.5 | 0.6% | Not determined |
| | and 6-OST-3 | | | | |
| 6 | Epi and 3-OST-1 | No 2-O- and 6-O- | 0.4 | 5.1% | Not determined |
| | | sulfations | | | |
| 7 | Epi, 6-OST-1, 6-OST-3 | No 2-O-sulfation | 1.3 | 29% | 25 nM |
| | and 3-OST-1 | | | | |
| 8 | Epi, 6-OST-1, 6-OST-3, | Very similar to the | 1.5 | 35% | 29 nM |
| | 3-OST-1 and 2-OST | HS isolated from | | | |
| | | tissues | | | |

¹These structural features are compared to heparin and HS isolated from natural sources.
²The percentage of the ³⁵S-labeled polysaccharide bound to AT-affinity column was determined incubating AT and the polysaccharides. The complex of AT and polysaccharide was captured by using ConA-agarose column.
³The binding affinity of the polysaccharides and AT was determined using affinity coelectrophoresis.
⁴Epi = epimerase

### Example 2

### Structural Characterization of Synthesized Polysaccharides

Sulfation Characteristics of the Polysaccharides. The synthesized HS-like compounds, i.e. compounds 1-8, were degraded and resolved using RPIP-HPLC as described hereinabove. The overall sulfation level among the products was between 0.9 and 1.4 nmoles of O-sulfo groups per μg of polysaccharide, namely 0.39 to 0.61 O-sulfo groups per disaccharide unit as measured based on the specific [³⁵S]radioactivity (Table 2). Compound 6 was an exception because it contained only 3-O-sulfation, which is an inherently low abundant sulfation type in the HS isolated from a natural source (Liu et al. (2002) Med. Res. Rev. 22, 1-25).

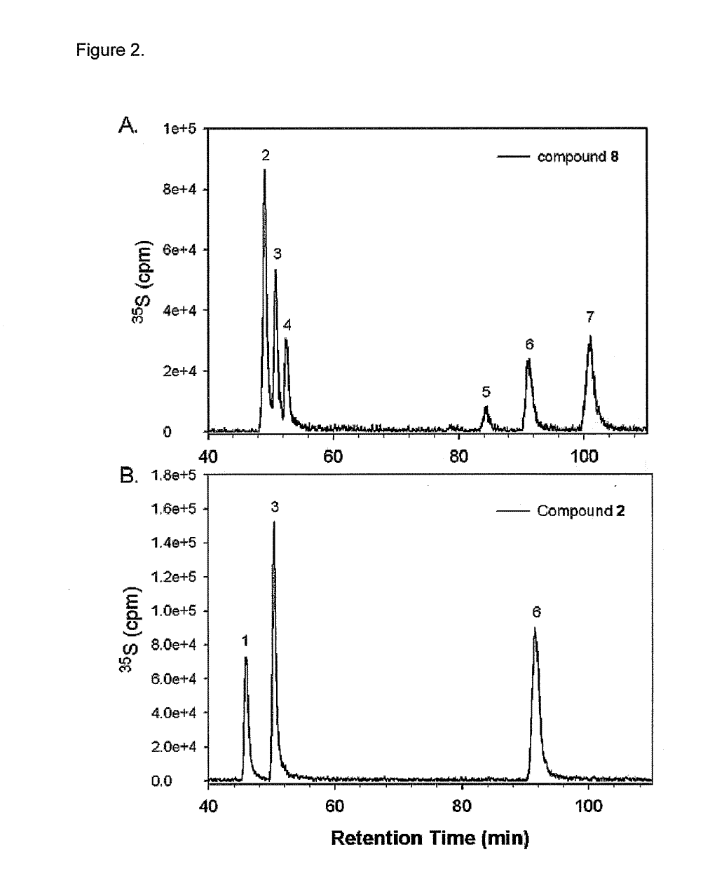

Disaccharide Analysis of the Polysaccharides. Among the synthesized AT-binding polysaccharides, compound 2 has the simplest structure because it should contain neither IdoUA nor 2-O-sulfo groups. To further elucidate the structure of the synthesized polysaccharides, disaccharide analysis of compound 2 was conducted and compared the disaccharide composition of compound 8. The resultant disaccharides were resolved on reversed-phase ion pairing (RPIP) HPLC (FIG. 2). Indeed, six disaccharides were observed in compound 8, while only three disaccharides were observed in compound 2. In addition, the ratio of the three disaccharides in compound 2, including GlcUA-AnMan3S, GlcUA-AnMan6S, and GlcUA-AnMan3S6S, is about 1:2:1 (Table 3), suggesting that this polysaccharide comprises a repeating tetrasaccharide unit with a structure of -GlcUA-GlcNS6S-GlcUAGlcNS3S±6S-. The results from disaccharide analysis also suggests that the IdoUA residue might not be essential for the polysaccharide to bind to AT.

TABLE 3
Compositional analysis of compounds 2 and 8

| | Compound 2 | Compound 8 |
|---|---|---|
| Disaccharides | (mol/mol %) | (mol/mol %) |
| GlcUA-AnMan3S | 24.7% | N.D.* |
| IdoUA2S-AnMan | N.D. | 35.9% |
| GlcUA-AnMan6S | 48.8% | 23.3% |
| IdoUA-AnMan6S | N.D. | 15.9% |
| IdoUA-AnMan3S6S | N.D. | 2.2% |
| GlcUA-AnMan3S6S | 26.2% | 8.9% |
| IdoUA2S-AnMan6S | N.D. | 13.4% |

*Not detectable

### Example 3

### Determination of the Biological Activities of the Synthesized HS-Like Compounds

The Binding of the Polysaccharides to AT. Subjecting the synthetic polysaccharides to an antithrombin (AT)-affinity column revealed that a significant portion of compounds 1, 2, 7, and 8 bound to AT, while compounds 3, 4, 5, and 6 showed much less or no binding to AT (Table 2). It is noted that 1, 2, 7, and 8 all carry 3-O- and 6-O-sulfo groups, while 3, 4, 5, and 6 either lacked 3-O-sulfo or 6-O-sulfo groups. Thus, both 3-O-sulfation and 6-O-sulfation of the glucosamine unit appear to play a role in binding to AT. In addition, 2-O-sulfation of IdoUA is not essential, as evidenced by the ability of compound 7 to bind to AT. The binding constants (K_{d}s) of the synthetic polysaccharides and AT were determined to be in the range of 27 to 57 nM (Table 2), which are very similar to that of previously characterized anticoagulant HS (Muñoz et al. (2006) Biochem. Biophys. Res. Commun. 339, 597-602).

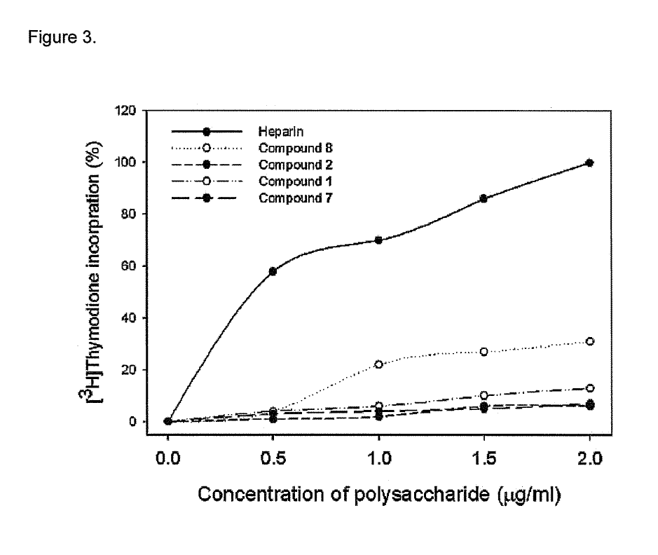

Cell Proliferative Activity of the Synthetic Polysaccharides. Heparin and HS form a ternary complex with fibroblast growth factors (FGFs) and fibroblast growth factor receptors (FGFR) to stimulate cell proliferation; however, this interaction is not related to the anticoagulant activity of HS. The instant methods provide for the separation of the anticoagulant activity and cell proliferation activity of the synthetic HS-like compounds using the BaF3 cells model system. BaF3 cells overexpressing FGFR1 normally depend on IL-3 for growth. In the absence of IL-3, the cell proliferation depends on the addition of both FGF and heparin or HS (Ornitz et al. (1996) J. Biol. Chem. 271, 15292-15297). The cells receiving heparin and compound 8 showed an increase in [³H]thymidine incorporation (FIG. 3). Compound 8 displayed a lower extent in stimulating BaF3 cell growth than that of heparin since its structure is closer to HS than heparin (Chen et al. (2005) J. Biol. Chem. 280, 42817-42825). It is also noted that compound 1 showed modest activity in stimulating cell proliferation. Compounds 2 and 7 had no detectable activity in stimulating cell proliferation, suggesting that the presently disclosed methods can provide for the separation of the anticoagulant activity and cell proliferation activity, such as by removing IdoUA2S and IdoUA residues from the polysaccharide.

The Anticoagulant Activity of the Synthesized Polysaccharides. Heparin achieves its anticoagulant activity by forming a 1:1 complex with AT, which inhibits the activities of factor Xa and thrombin (Rosenberg et al. (1997) J. Clin. Invest 99, 2062-2070). The synthetic HS-like compounds displayed anticoagulant activity as measured by anti-Xa and anti-IIa activities (Table 4). The IC₅₀ values of compounds 1, 2, 7, and 8 for Xa are 2 to 2.5 fold higher than that of unfractionated heparin, while about 2-fold lower than that of LOVENOX®, a low molecular weight heparin drug manufactured by Aventis (Aventis, Bridgewater, N.J.). A similar trend was observed for the anti-1a activity of these compounds. The N-sulfo heparosan 6-O-sulfate was used as a negative control showing no inhibitory effect on the activity of Xa and IIa.

TABLE 4
Anti-Xa and Anti-IIa activities of the synthetic polysaccharides

| | Anti-Xa activity, IC₅₀ | Anti-IIa activity, IC₅₀ |
|---|---|---|
| Compounds | (ng/ml) | (ng/ml) |
| Heparin | 25 | 8 |
| 1 | 50 | 18 |
| 2 | 50 | 20 |
| 7 | 45 | 16 |
| 8 | 40 | 15 |
| N-sulfo heparosan | >5,000 | >3,000 |
| 6-O-sulfate^{a} | | |
| LOVENOX ®^{b} | 90 | 50 |

^{a}The synthesis of N-sulfo heparosan 6-O-sulfate is displayed in Supplementary FIG. 7.
^{b}LOVENOX ® was purchased from a local pharmacy.

### Example 4

### Characterization of the Synthetic HS-Like Compounds without Iduronic Acid

As noted above, compound 2 has no IdoUA residue but has high binding affinity to AT and strong anticoagulant activity, which appears to be contradictory to previously published results. The IdoUA residue has been implicated to be essential for the binding to AT because it provides the conformational flexibility (Petitou et al. (2004) Angew. Chem. Int Ed. 43, 3118-3133). In the pentasaccharides, the IdoUA residue exists in either a chair (¹C₄) or a skew boat (²S₀) conformation (Petitou et al., 2004). Furthermore, the unique chair conformation (²S₀) of IdoUA2S in a synthetic pentasaccharide has been proven to be essential for the binding to AT (Das et al. (2001) Chemistry 7, 4821-4834). However, in light of the evidence present herein, it is believed that necessity in the conformational flexibility for AT-binding affinity is size dependent. To test this hypothesis, the AT-binding efficiency of HS-like compounds prepared using the mixture of enzymes with or without the capability of synthesizing IdoUA2S were compared. In one preparation, the oligosaccharides (for the synthesis of oligosaccharides without the IdoUA2S residue) were incubated with the mixture of enzymes containing 6-OST-1,6-OST-3 and 3-OST-1. In another preparation, the oligosaccharides (for the synthesis of oligosaccharides with the IdoUA2S residue) were incubated with a mixture of enzymes containing epimerase and 2-OST as well as 6-OST-1,6-OST-3 and 3-OST-1.

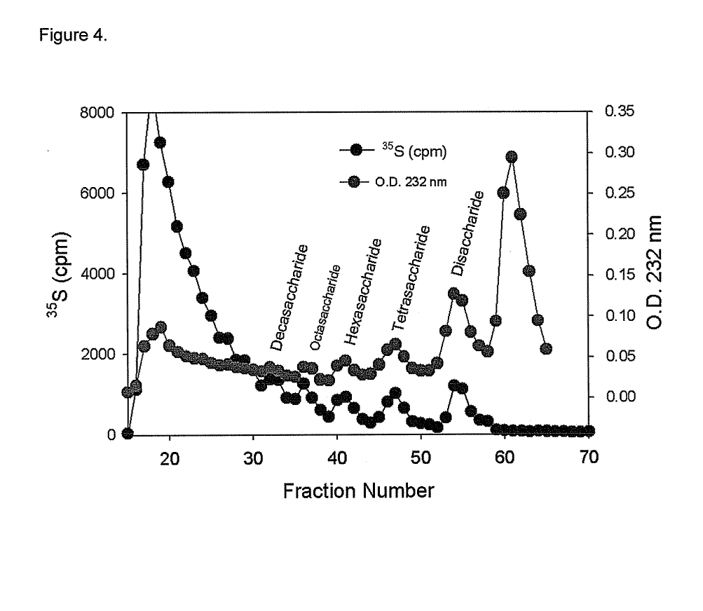

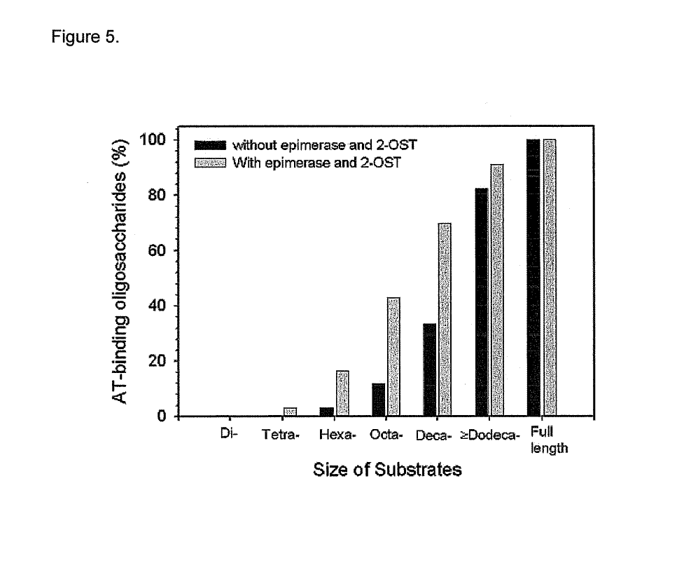

The oligosaccharide substrates with structures of ΔUA-(GlcNS-GlcUA)n-GlcNS-, where n=0, 1, 2, 3, 4 and ≧5, were prepared by subjecting N-sulfo heparosan to partial depolymerization with heparin lyase III followed by fractionation on gel permeation chromatography (FIG. 4). After the modifications, the AT-binding ³⁵S-labeled oligosaccharides were captured by the AT-affinity column, and the data is shown in FIG. 5.

In the absence of epimerase and 2-OST, ³⁵S-labeled octa- and decasaccharides showed substantial binding to AT, while ³⁵S-labeled hexasaccharide showed very low yield (FIG. 5). In contrast, using the same oligosaccharide substrates in the presence of epimerase and 2-OST, ³⁵S-labeled hexasaccharides displayed significant binding to AT. As such, it appears that the amount of AT-binding hexasaccharides and octasaccharides is about 4 to 5 fold higher than when the substrates were modified with a mixture of enzymes containing epimerase and 2-OST. These results suggest that the contribution of the IdoUA2S on the AT-binding affinity could be less essential when the size of the HS-like compound is larger than an octasaccharide.

Synthesis of HS-like compounds using epimerase results in compounds containing a mixture of IdoUA and GlcUA because the reaction catalyzed by epimerase is reversible (Li et al. (1997) J. Biol. Chem. 272, 28158-28163). Consequently, the product has greater structural heterogeneity, increasing the possibility of untoward effects of an anticoagulant drug. Anticoagulant HS-like compounds without the IdoUA residues can decrease the structural heterogeneity and reduce the complexity in the synthesis of HS-based anticoagulant drugs. Further, because HS is also involved in tumor growth and viral infections, the enzyme-based synthetic approach can be used to prepare the HS structures displaying anticancer and antiviral activities.

### Example 5

### Large-Scale Production of HS-Like Compounds without Iduronic Acid Using an Enzyme-Based Combinatorial Approach

Because of its activity and reduced structural heterogeneity, compound 2 was synthesized on a larger scale. As before, compound 2 was synthesized from deacetylated heparosan using sequential modifications with N-sulfotransferase (NST), 6-OST-1,6-OST-3 and 3-OST-1 as illustrated in FIG. 6. In this synthesis, p-nitrophenol sulfate (PNPS) replaced PAPS as a sulfo donor in the PAPS regeneration system (Chen et al., (2005) J. Biol. Chem. 280, 42817-42825; Burkart et al. (2000) J. Org. Chem. 65, 5565-5574). Disaccharide analysis on the intermediate compounds, including N-sulfo heparosan and N-sulfo heparosan 6-O-sulfate, revealed that N-sulfation reached 75.6% and 6-O-sulfation reached 85% (Table 5). The 3-O-sulfation carried out by 3-OST-1 went to about 90% completion, and was estimated to be 0.5 3-O-sulfo groups/disaccharide based on the modification using [³⁵S]PAPS. The final product, renamed as Recomparin (for recombinant heparin), showed anti-Xa activity at the IC₅₀ value of 25 ng/ml, which is very close to the value of heparin at 19 ng/ml (FIG. 7).

TABLE 5
Summary of the disaccharide analysis of N-sulfo
heparosan and N-sulfo heparosan 6-O-sulfate

| | Anti-Xa activity, IC₅₀ | Anti-IIa activity, IC₅₀ |
|---|---|---|
| Compounds | (ng/ml) | (ng/ml) |
| Heparin | 25 | 8 |
| 1 | 50 | 18 |
| 2 | 50 | 20 |
| 7 | 45 | 16 |

### Discussion of Examples 1-5

HS is a major component on the mammalian cell surface and in the extracellular matrix with a wide range of biological functions. There is considerable interest for developing HS-based drugs to inhibit tumor growth, bacterial viral infections and bacterial infections and modulate inflammatory responses, including anticoagulation, antiviral, and anticancer activities. Sulfo group-containing saccharide sequences dominate the specificity of the functions of heparin and HS. Thus, the synthesis of a polysaccharide with the appropriate positioning of these functional groups to carry out its unique biological activity is desirable. The presently disclosed subject matter shows that an enzymatic approach is a viable method for generating the polysaccharides with different sulfation patterns and biological functions. The synthesis can be conducted in parallel to produce a large number of compounds by varying the levels of individual enzymes.

In summary, this present disclosure provides a novel approach to preparing HS-like compounds with unique sulfation patterns. The presently disclosed subject matter provides methods for enzymatic sulfation and preparation of HS-like compounds with distinct biological activities. The presently disclosed methods demonstrate the capability of using a collection of HS biosynthetic enzymes to synthesize HS-like compounds with selected biological activities. The results presented herein also show that the enzyme-based combinatorial approach to synthesizing HS-like compounds is fully capable of synthesizing milligram scales of these anticoagulant polysaccharides.

The generation of a HS library allows for the investigation of the contribution of each of the sulfo groups to the biological function(s) of HS-like compounds. Utilizing this enzyme-based combinatorial HS biosynthetic system, one of ordinary skill in the art can prepare a variety of HS structures by systematically including and/or excluding certain enzymes. Unlike chemical sulfonation approaches, the instant methods permit the synthesis of HS-like compounds that are restricted with certain types of sulfations due to the high substrate specificities of sulfotransferases. More particularly, this approach permits the synthesis of biologically active HS-like compounds that can mimic the action of HS under physiological conditions.

As stated hereinabove, employing the enzyme-based combinatorial approach to synthesize HS-like compounds revealed that anticoagulant polysaccharides do not require the IdoUA residue. Polysaccharides without an IdoUA residue, i.e. IdoUA-free HS-like compounds, displayed strong binding affinity to antithrombin and high anti-Xa and anti-IIa activities. Further, these IdoUA-free HS-like compounds have substantially reduced activity in promoting cell proliferation. In some embodiments, a substantially reduced activity in promoting cell proliferation can comprise a cell proliferation promoting activity of 5% or less of the cell proliferation promoting activity of a heparin counterpart. Stated another way, IdoUA-free HS-like compounds having substantially reduced activity in promoting cell proliferation can have a 95% or greater reduction in cell proliferation promoting activity as compared to a non-IdoUA-free HS-like compound. As such, the presently disclosed subject matter provides for the synthesis of functionally specific anticoagulant polysaccharides.

Employing the instant methods can also provide for the characterization of the precise structure of the anticoagulant structure without IdoUA. Characterizing the structure of an anticoagulant without IdoUA can simplify the synthesis of new anticoagulant drugs with reduced untoward effects. Due to the versatility and flexibility of the enzyme-based combinatorial synthetic methods disclosed herein, it is believed that they are a useful tool for identifying the lead structures for the development of HS-based therapeutic agents. The presently disclosed methods can aid the exploration of therapeutic applications for HS-like compounds. In addition, enzymatic synthesis of anticoagulant heparin can provide an anticoagulant drug with reduced side effects.

### REFERENCES

The references listed below as well as all references cited in the specification are incorporated herein by reference to the extent that they supplement, explain, provide a background for or teach methodology, techniques and/or compositions employed herein.
Aikawa et al., (1999) J. Biol. Chem. 274, 2690.
Aikawa, J.-I., et al., (2001) J. Biol. Chem. 276, 5876-5882.
Alexander, C. M., et al., (2000) Nat. Genet. 25, 329-332.
Altschul et al. (1990) J Mol Biol 215, 403-410.
Atha, D. H., Lormeau, J.-C., Petitou, M., Rosenberg, R. D., and Choay, J. (1985) Biochemistry 24, 6723-6729.
Avci, F. Y., Karst, N. A., and Linhardt, R. J. (2003) Curr. Pharm. Des. 9, 2323-2335.
Balagurunathan, K., Beeler, D. L., Lech, M., Wu, Z. L., and Rosenberg, R. D. (2003) J. Biol. Chem. 278, 52613-52621.
Balagurunathan, K., Lech, M. Z., Beeler, D. L., Wu, Z. L., and Rosenberg, R. D. (2003) Nat. Biotechnol. 21, 1343-1346.
Bernfield, M., et al., (1999) Annu. Rev. Biochem. 68, 729-777.
Bjornsson, S. (1993) Anal. Biochem. 210, 282-291.
Bowman et al., (1999) Chem. Biol. 6, R9-R22.
Burkart, M. D., Izumi, M., Chapman, E., Lin, C., and Wong, C. (2000) J. Org. Chem. 65, 5565-5574.
Capila, I., and Linhardt, R. J. (2002) Angew. Chem. Int Ed. 41, 390-412.
Carfi, A., et al., (2001) Mol. Cell 8:169-179.
Chen et al., (1992) Protein Expression Purif. 3, 421-6.
Chen, J., Duncan, M. B., Carrick, K., Pope, M., and Liu, J. (2003) Glycobiology 13, 785-794.
Chen et al., (2005) J. Biol. Chem. 280, 42817-42825.
Conrad, H. (1998) Heparin-binding Proteins, Academic Press, San Diego, Calif.
Copeland et al., (2008) Biochemistry 47: 5774-5783.
Das et al. (2001) Chemistry 7, 4821-4834.
Dementiev, A., Petitou, M., Herbert, J.-M., and Gettins, P. G. (2004) Nat Struct Biol. 11, 867-863.
Dooley (1998) Chemico-Biol. Interact. 109, 29.
Duncan, M. B., Chen, J., Krise, J. P., and Liu, J. (2004) Biochim. Biophys. Acta 1671, 34-43.
Edavettal, S. C., Lee, K. A., Negishi, M., Linhardt, R. J., Liu, J., and Pedersen, L. C. (2004) J. Biol. Chem. 279, 25789-25797.
Esko, J. D., and Lindahl, U. (2001) J. Clin. Invest. 108:169-173.
Esko, J. D., and Selleck, S. B. (2002) Annu. Rev. Biochem. 71, 435-471.
Falany (1997) FASEB J. 11, 1-2.
Feyerabend et al. (2006) Nat. Chem. Biol. 2, 195-196.
Fukuta et al. (1998) Biochim. Biophys. Act. 1399, 57.
Fuster et al. (2005) Nat. Rev. Cancer 5, 526-542.
Gama et al. (2006) Nat. Chem. Biol. 2, 467-473.
Gribskov et al. (1986) Nuc Acids Res 14(1), 327-334.
Guo et al. (1994) Chem.-Biol. Interact 92, 25-31.
Habuchi et al., (1998) J. Biol. Chem. 273, 9208.
Habuchi, H., et al., (2000) J. Biol. Chem. 275, 2859-2868.
Kakuta et al. (2003) Biochem. Soc. Trans. 31 (pt2), 331-334.
Kreuger et al. (2006) J. Cell Biol. 174, 323-327.
Krummenacher, C., et al., (1999) J. Virol. 73, 8127-8137.
Kuberan, B., Beeler, D. L., Lawrence, R., Lech, M., and Rosenberg, R. (2003) J.
Am. Chem. Soc. 125, 12424-12425.
Kyte et al. (1982) J Mol Biol 157, 105.
Lee, M. K., and Lander, A. D., (1991) Proc. Natl. Acad. Sci. USA 88, 2768-2772.
Li et al. (1997) J. Biol. Chem. 272, 28158-28163
Lin et al., (1995) J. Am. Chem. Soc. 117, 8031.
Lin et al. (1998) Anal. Biochem. 264, 111-117.
Lindahl, U., et al., (1998) J. Biol. Chem. 273, 24979-24982.
Lindahl, U., Li, J., Kusche-Gullberg, M., Saimivirta, M., Alaranta, S., Veromaa, T., Emies, J., Roberts, I., Taylor, C., Oreste, P., Zoppetti, G., Naggi, A., Torri, G., and Casu, B. (2005) J. Med. Chem. 48, 349-352.
Linhardt, R. J. (2003) J. Med. Chem. 46, 2551-2564.
Liu, J., and Thorp, S. C. (2002) Med. Res. Rev. 22, 1-25.
Liu, J., et al., (1996) J. Biol. Chem. 271, 27072-27082.
Liu, J., et al., (1999) J. Biol. Chem. 274, 38155-38162.
Liu, J., et al., (1999) J. Biol. Chem. 274, 5185-5192.
Liu, J., Shworak, N. W., Sinay, P., Schwartz, J. J., Zhang, L., Fritze, L. M. S., and Rosenberg, R. D. (1999) J. Biol. Chem. 274, 5185-5192.
Liu, J., Shriver, Z., Blaiklock, P., Yoshida, K., Sasisekharan, R., and Rosenberg, R. D. (1999) J. Biol. Chem. 274, 38155-38162.
Liu, J., et al., (2002) J. Biol. Chem. 277, 33456-33467.
Liu et al. (2007) Appl. Microbiol. Biotechnol. 74, 263-272.
Marcus et al. (1980) Aial. Biochem. 107, 296.
Marshall et al., (1997) J. Biol. Chem. 272, 9153-9160.
Marshall et al., (1998) Chem.-Biol. Interact 109, 107-116.
Mazany et al., (1998) Biochim. Biophys. Act 1407, 92.
Moon, A., Edavettal, S. C., Krahn, J. X., Munoz, E. M., Negishi, M., Linhardt, R. J., Liu, J., and Pedersen, L. C. (2004) J. Biol. Chem. 279, 45185-45193.
Muñoz et al. (2006) Biochemistry 45, 5122-5128.
Muñoz et al. (2006) Biochem. Biophys. Res. Commun. 339, 597-602
Nastuk et al. (1998) J. Neuroscience 18, 7167.
Needleman et al. (1970) J Mol Biol 48, 443.
Ong et al., (1998) J. Biol. Chem. 273, 5190.
Ornitz, D. M., Xu, J., Colvin, J. S., McEwen, D. G., MacArthur, C. A., Coulier, F., Gao, G., and Goldfarb, M. (1996) J. Biol. Chem. 271, 15292-15297.
Ouyang et al., (1998) J. Biol. Chem. 273, 24770.
Ozawa et al., (1990) Nucleic Acids Res. 18, 4001z.
Petitou, M., Herault, L.-P., Bernat, A., Driguez, P.-A., Duchaussoy, P., Lormeau, J.-C., and Herbert, J.-M. (1999) Nature 398, 417-422.
Petitou, M., and van Boeckel, C. A. A. (2004) Angew. Chem. Int Ed. 43, 3118-3133.
Reizes, O., et al., (2001) Cell 106:105-116.
Rosenberg, R. D., et al., (1997) J. Clin. Invest 99, 2062-2070.
Saeki et al. (1998) J. Biochem. 124, 55.
Sambrook et al. (2001) Molecular Cloning: A Laboratory Manual, Third Edition, Cold Spring Harbor Laboratory Press, Cold Spring Harbor, N.Y.
Sasisekharan, R., Shriver, Z., Venkataraman, G., and Narayanasami, U. (2002) Nat. Rev. Cancer 2, 521-528.
Schwartz et al. (1979) Nuc Acids Res 6(2), 745-755.
Shively, J. E., and Conrad, H. E. (1976) Biochemistry 15, 3932-3942.
Shriver et al. (2004) Nat. Rev. Drug Discov. 863-873.
Shukla, D., et al. (1999) Cell 99, 13-22.
Shukla, D., and Spear, P. G. (2001) J. Clin. Invest 108, 503-510.
Shworak, N. W., et al., (1997) J. Biol. Chem. 272, 28008-28019.
Smith et al. (1981) Adv Appl Math 2, 482.
Thompson et al. (1994) Nucleic Acids Res 22(22), 4673-4680.
U.S. Pat. No. 6,255,088.
U.S. Pat. No. 4,554,101.
Uchimura et al. (1998) J. Biol. Chem. 273, 22577.
Vann et al., (1981) Eur. J. Biochem. 116, 359-364.
Wethmur & Davidson (1968) J Mol Biol 31, 349-370.
Willis, S. H., et al., (1998) J. Virol. 72, 5938-5947.
WuDunn, D., and Spear, P. G. (1989) J. Virol. 63, 52-58.
Yang et al. (1996) Protein Expression Purif. 8, 423-429.
Yang et al., (1997) Protein Eng. 10, 70.
Yang et al., (1998) Chem.-Biol. Interact 109, 129-135.
Yoshinari et al., (1998) J. Biochem. 123, 740.
Zhang, L., Beeler, D. L., Lawrence, R., Lech, M., Liu, J., Davis, J. C., Shriver, Z., Sasisekharan, R., and Rosenberg, R. D. (2001) J. Biol. Chem. 276, 42311-42321.
Zhang, L., Lawrence, R., Schwartz, J. J., Bai, X., Wei, G., Esko, J. D., and Rosenberg, R. D. (2001) J. Biol. Chem. 276, 28806-28813.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. An iduronic acid (IdoUA)-free heparan sulfate (HS)-like compound comprising the following structure: 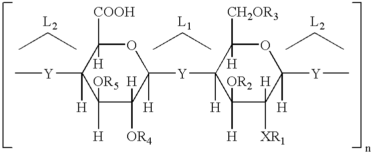
wherein X is NH or O, R₁ is H, SO₃H or CH₃, R₂ is SO₃H, R₃ is H or SO₃H, R₄ is H, SO₃H, CH₃ or CH₂CH₃, R₅ is H, SO₃H, CH₃ or CH₂CH₃, R₆ is COOH, COOCH₃, COOC₂H₅, CH₂OSO₃H, or CH₂SO₃H, L₁ is an alpha or beta linkage, L₂ is an alpha or beta linkage, Y is O or N, and n is ≧4.

2. The IdoUA-free HS-like compound of claim 1, wherein X is NH, R₁ is SO₃H, R₂ is SO₃H, R₃ is SO₃H, R₄ is H or SO₃H, R₅ is H, L₁ is an alpha or a beta linkage, L₂ is an alpha or a beta linkage, and n is ≧4.

3. The IdoUA-free HS-like compound of claim 2, wherein L₁ is an alpha linkage and L₂ is a beta linkage.

4. The IdoUA-free HS-like compound of claim 2, having a binding affinity to antithrombin ranging from about 20 to about 60 nM.

5. The IdoUA-free HS-like compound of claim 2, having an anti-Xa activity ranging from about 10 to about 500 ng/ml.

6. The IdoUA-free HS-like compound of claim 2, having an anti-IIa activity ranging from about 5 to about 200 ng/ml.

7. The IdoUA-free HS-like compound of claim 2, wherein the HS-like compound has substantially reduced cell proliferation activity as compared to a HS-like compound with iduronic acid residues.

8. The IdoUA-free HS-like compound of claim 1, wherein R₄ is selected from the group consisting of CH₃, CH₂CH₃ and SO₃H and R₅ is selected from the group consisting of CH₃, CH₂CH₃ and SO₃H.

9. The IdoUA-free HS-like compound of claim 1, wherein X is O and R₁ is SO₃H.

10. The IdoUA-free HS-like compound of claim 1, wherein L₁ is an alpha linkage and L₂ is an alpha linkage.

11. The IdoUA-free HS-like compound of claim 1, wherein L₁ is a beta linkage and L₂ is a beta linkage.

12. A method of synthesizing an IdoUA-free HS-like compound, comprising incubating a polysaccharide substrate with a sulfo donor compound and an enzyme mixture, the enzyme mixture comprising O-sulfotransferase (OST) enzymes 6-OST-1,6-OST-3 and 3-OST-1 and substantially no epimerase enzyme, wherein synthesis of the IdoUA-free HS-like compound from the polysaccharide substrate is accomplished.

13. The method of claim 12, wherein the polysaccharide substrate is selected from the group consisting of N-sulfo heparosan and chemically desulfated N-sulfated heparin.

14. The method of claim 12, wherein the OST enzymes are recombinant OST enzymes.

15. The method of claim 14, wherein the recombinant OST enzymes are produced in a bacterial expression system.

16. The method of claim 12, wherein the sulfo donor compound comprises a compound capable of donating a sulfonate or sulfuryl group.

17. The method of claim 12, wherein the polysaccharide substrate is partially sulfated prior to incubation.

18. The method of claim 12, further comprising:
(a) providing a reaction mixture comprising adenosine 3′,5′-diphosphate (PAP), a PAPS regenerating enzyme and a sulfo donor compound; and
(b) incubating the reaction mixture for a time period sufficient to catalyze the production of 3′-phosphoadenosine 5′-phosphosulfate (PAPS) from the PAP by the PAPS regenerating enzyme utilizing the sulfo donor compound as a sulfo substrate,
whereby the sulfo donor compound is provided.

19. The method of claim 18, wherein the PAPS regenerating enzyme is an arylsulfotransferase.

20. The method of claim 19, wherein the arylsulfotransferase is AST-IV.

21. The method of claim 18, wherein the PAPS regenerating enzyme is an estrogen sulfotransferase.

22. The method of claim 18, wherein the sulfo donor compound is an aryl sulfate compound.

23. The method of claim 22, wherein the aryl sulfate compound is p-nitrophenol sulfate (PNPS).

24. The method of claim 18, wherein the time period is from about 1 minute to about 30 minutes.

25. The method of claim 18, wherein the OST enzymes further comprise 2-OST.

26. The method of claim 25, wherein the 2-OST enzyme is a recombinant OST enzyme.

27. The method of claim 26, wherein the recombinant OST enzyme is produced in a bacterial expression system.

28. A method of synthesizing a library of HS-like compounds, the method comprising incubating polysaccharide substrates with a sulfo donor compound and one or more combinations of biosynthetic enzymes, the biosynthetic enzymes comprising epimerases, N-sulfotransferases and O-sulfotransferases.

29. The method of claim 28, wherein the polysaccharide substrate is N-sulfo heparosan.

30. The method of claim 28, wherein the O-sulfotransferases are selected from the group consisting of 2-OSTs, 3-OSTs and 6-OSTs.

31. The method of claim 28, wherein the O-sulfotransferase enzymes are recombinant OST enzymes.

32. The method of claim 31, wherein the recombinant OST enzymes are produced in a bacterial expression system.

33. The method of claim 28, wherein the polysaccharide substrate is partially sulfated prior to incubation.

34. The method of claim 28, further comprising:
(c) providing a reaction mixture comprising adenosine 3′,5′-diphosphate (PAP), a PAPS regenerating enzyme and a sulfo donor compound; and
(d) incubating the reaction mixture for a time period sufficient to catalyze the production of 3′-phosphoadenosine 5′-phosphosulfate (PAPS),
whereby the sulfo donor compound is provided.

35. The method of claim 34, wherein the PAPS regenerating enzyme is an arylsulfotransferase.

36. The method of claim 35, wherein the arylsulfotransferase is AST-IV.

37. The method of claim 34, wherein the PAPS regenerating enzyme is an estrogen sulfotransferase.

38. The method of claim 34, wherein the sulfo donor compound is an aryl sulfate compound.

39. The method of claim 38, wherein the aryl sulfate compound is p-nitrophenol sulfate (PNPS).

40. The method of claim 34, wherein the time period is from about 1 minute to about 30 minutes.

41. The method of claim 34, wherein the polysaccharide substrate is a chemically desulfated N-sulfated heparin.

42. A method of determining a mechanism of activity of HS-like compounds, the method comprising:
(e) synthesizing one or more HS-like compounds by incubating one or more polysaccharide substrates with a sulfo donor compound and one or more combinations of biosynthetic enzymes, the biosynthetic enzymes comprising epimerases, N-sulfotransferases and O-sulfotransferases;
(f) subjecting one or more of the HS-like compounds to a test for an activity; and
(g) determining a mechanism of activity of the HS-like compounds based on one or more results of the one or more HS-like compounds.

43. The method of claim 42, wherein the polysaccharide substrate is selected from the group consisting of N-sulfo heparosan and chemically desulfated N-sulfated heparin.

44. The method of claim 42, wherein the O-sulfotransferases are selected from the group consisting of 2-OSTs, 3-OSTs and 6-OSTs.

45. The method of claim 42, wherein the O-sulfotransferase enzymes are recombinant OST enzymes.

46. The method of claim 45, wherein the recombinant OST enzymes are produced in a bacterial expression system.

47. The method of claim 42, wherein the polysaccharide substrate is partially sulfated prior to incubation.

48. The method of claim 42, wherein the test is for an activity selected from the group consisting of anti-thrombin binding activity, anti-Xa activity, anti-IIa activity, cell proliferation stimulation activity, cell growth stimulation activity, activated partial thromboplastin time, prothrombin time and combinations thereof.

49. The method of claim 42, further comprising:
(h) providing a reaction mixture comprising adenosine 3′,5′-diphosphate (PAP), a PAPS regenerating enzyme and a sulfo donor compound; and
(i) incubating the reaction mixture for a time period sufficient to catalyze the production of 3′-phosphoadenosine 5′-phosphosulfate (PAPS),
whereby the sulfo donor compound is provided.

50. The method of claim 49, wherein the PAPS regenerating enzyme is an arylsulfotransferase.

51. The method of claim 50, wherein the arylsulfotransferase is AST-IV.

52. The method of claim 49, wherein the PAPS regenerating enzyme is an estrogen sulfotransferase.

53. The method of claim 49, wherein the sulfo donor compound is an aryl sulfate compound.

54. The method of claim 53, wherein the aryl sulfate compound is p-nitrophenol sulfate (PNPS).

55. The method of claim 49, wherein the time period is from about 1 minute to about 30 minutes.


SEQUENCE LISTING (8 sequences)

SEQ ID NO: 1 - protein, 356 aa, Mus musculus
MGLLRIMMPPKLQLLAVVAFAVAMLFLENQIQKLEESRAKLERAIARHEVREIEQRHTMDGPRQDATLDEEEDIIIIYNRVPKTASTSFTNIAYDLCAKNRYHVLHINTTKNNPVMSLQDQVRFVKNITTWNEMKPGFYHGHISYLDFAKFGVKKKPIYINVIRDPIERLVSYYYFLRFGDDYRPGLRRRKQGDKKTFDECVAEGGSDCAPEKLWLQIPFFCGHSSECWNVGSRWAMDQAKSNLINEYFLVGVTEELEDFIMLLEAALPRVFRGATDLYRTGKKSHLRKTTEKKLPTKQTIAKLQQSDIWKMENEFYEFALEQFQFIRAHAVREKDGDLYILAQNFFYEKIYPKSN

SEQ ID NO: 2 - protein, 307 aa, Homo sapiens
MAALLLGAVLLVAQPQLVPSRPAELGQQELLRKAGTLQDDVRDGVAPNGSAQQLPQTIIIGVRKGGTRALLEMLSLHPDVAAAENEVHFFDWEEHYSHGLGWYLSQMPFSWPHQLTVEKTPAYFTSPKVPERVYSMNPSIRLLLILRDPSERVLSDYTQVFYNHMQKHKPYPSIEEFLVRDGRLNVDYKALNRSLYHVHMQNWLRFFPLRHIHIVDGDRLIRDPFPEIQKVERFLKLSPQINASNFYFNKTKGFYCLRDSGRDRCLHESKGRAHPQVDPKLLNKLHEYFHEPNKKFFELVGRTFDWH

SEQ ID NO: 3 - protein, 406 aa, Homo sapiens
MAPPGPASALSTSAEPLSRSIFRKFLLMLCSLLTSLYVFYCLAERCQTLSGPVVGLSGGGEEAGAPGGGVLAGGPRELAVWPAAAQRKRLLQLPQWRRRRPPAPRDDGEEAAWEEESPGLSGGPGGSGAGSTVAEAPPGTLALLLDEGSKQLPQAIIIGVKKGGTRALLEFLRVHPDVRAVGAEPHFFDRSYDKGLAWYRDLMPRTLDGQITMEKTPSYFVTREAPARISAMSKDTKLIVVVRDPVTRAISDYTQTLSKRPDIPTFESLTFKNRTAGLIDTSWSAIQIGIYAKHLEHWLRHFPIRQMLFVSGERLISDPAGELGRVQDFLGLKRIITDKHFYFNKTKGFPCLKKAEGSSRPHCLGKTKGRTHPEIDREVVRRLREFYRPFNLKFYQMTGHDFGWDG

SEQ ID NO: 4 - protein, 390 aa, Homo sapiens
MGQRLSGGRSCLDVPGRLLPQPPPPPPPVRRKLALLFAMLCVWLYMFLYSCAGSCAAAPGLLLLGSGSRAAHDPPALATAPDGTPPRLPFRAPPATPLASGKEMAEGAASPEEQSPEVPDSPSPISSFFSGSGSKQLPQAIIIGVKKGGTRALLEFLRVHPDVRAVGAEPHFFDRSYDKGLAWYRDLMPRTLDGQITMEKTPSYFVTREAPARISAMSKDTKLIVVVRDPVTRAISDYTQTLSKRPDIPTFESLTFKNRTAGLIDTSWSAIQIGIYAKHLEHWLRHFPIRQMLFVSGERLISDPAGELGRVQDFLGLKRIITDKHFYFNKTKGFPCLKKAEGSSRPHCLGKTKGRTHPEIDREVVRRLREFYRPFNLKFYQMTGHDFGWD

SEQ ID NO: 5 - protein, 401 aa, Mus musculus
MVERASKFVLVVAGSACFMLILYQYAGPGLSLGAPGGRVPPDDLDLFPTPDPHYEKKYYFPVRELERSLRFDMKGDDVIVFLHIQKTGGTTFGRHLVQNVRLEVPCDCRPGQKKCTCYRPNRRETWLFSRFSTGWSCGLHADWTELTNCVPGVLDRRDPAGLRSPRKFYYITLLRDPVSRYLSEWRHVQRGATWKTSLHMCDGRTPTPEELPPCYEGTDWSGCTLQEFMDCPYNLANNRQVRMLADLSLVGCYNLSFIPESKRAQLLLESAKKNLRGMAFFGLTEFQRKTQYLFERTFNLKFIRPFMQYNSTRAGGVEVDEDTIRHIEELNDLDMQLYDYAKDLFQQRYQYKRQLERREQRLRNREERLLHRSKEALPREDPEEPGRVPTEDYMSHIIEKW

SEQ ID NO: 6 - protein, 506 aa, Mus musculus
MDEKSNKLLLALVMLFLFAVIVLQYVCPGTECQLLRLQAFSSPVPDPYRSEDESSARFVPRYNFSRGDLLRKVDFDIKGDDLIVFLHIQKTGGTTFGRHLVRNIQLEQPCECRVGQKKCTCHRPGKRETWLFSRFSTGWSCGLHADWTELTSCVPAVVDGKRDARLRPSRWRIFQILDGTSKDRWGSSNFNSGANSPSSTKPRSTSKSGKNFHYITILRDPVSRYLSEWRHVQRGATWKASLHVCDGRPPTSEELPSCYTGDDWSGCPLKEFMDCPYNLANNRQVRMLSDLTLVGCYNLSVMPEKQRNKVLLESAKSNLKHMAFFGLTEFQRKTQYLFEKTFNMNFISPFTQYNTTRASSVEINEEIQKRIEGLNFLDMELYSYAKDLFLQRYQFMRQKEHQDARRKRQEQRKFLKGRFLQTHFQSQSQGQSQSQSPGQNLSQNPNPNPNQNLTQNLSHNLTPSSNPNSTQRENRGSQKQGSGQGQGDSGTSNGTNDYIGSVETWR

SEQ ID NO: 7 - protein, 470 aa, Mus musculus
MDERFNKWLLTPVLTFLFVVIMYQYVSPSCTSSCTNFGEQLRSGEARPPAVPSPARRAQAPLDEWERRPQLPPPPRGPPEGSRGVAAPEDEDEDPGDPEEEEEEEEEEPDPEAPENGSLPRFVPRFNFTLKDLTRFVDFNIKGRDVIVFLHIQKTGGTTFGRHLVKNIRLEQPCSCKAGQKKCTCHRPGKKETWLFSRFSTGWSCGLHADWTELTNCVPAIMEKKDCPRNHSHTRNFYYITMLRDPVSRYLSEWKHVQRGATWKTSLHMCDGRSPTPDELPTCYPGDDWSGVSLREFMDCSYNLANNRQVRMLADLSLVGCYNLTFMNESERNTILLQSAKNNLKNMAFFGLTEFQRKTQFLFERTFNLKFISPFTQFNITRASNVDINDGARQHIEELNFLDMQLYEYAKDLFQQRYHHTKQLEHQRDRQKRREERRLQREHRAHRWPKEDRAMEGTVTEDYNSQVVRW

SEQ ID NO: 8 - protein, 291 aa, Rattus norvegicus
MEFSRPPLVHVKGIPLIKYFAETIGPLQNFTAWPDDLLISTYPKSGTTWMSEILDMIYQGGKLEKCGRAPIYARVPFLEFKCPGVPSGLETLEETPAPRLLKTHLPLSLLPQSLLDQKVKVIYIARNAKDVVVSYYNFYNMAKLHPDPGTWDSFLENFMDGEVSYGSWYQHVKEWWELRHTHPVLYLFYEDIKENPKREIKKILEFLGRSLPEETVDSIVHHTSFKKMKENCMTNYTTIPTEIMDHNVSPFMRKGTTGDWKNTFTVAQNERFDAHYAKTMTDCDFKFRCEL